(19) United States
(12) Patent Application Publication
(10) Pub. No.: US 2013/0203735 A1
(43) Pub. Date: Aug. 8, 2013

(54) CAPROLACTAM MGLUR5 RECEPTOR MODULATORS

(71) Applicants: John T. Sisko, Landsdale, PA (US); Thomas J. Tucker, North Wales, PA (US); Robert M. Tynebor, Harleysville, PA (US)

(72) Inventors: John T. Sisko, Landsdale, PA (US); Thomas J. Tucker, North Wales, PA (US); Robert M. Tynebor, Harleysville, PA (US)

(21) Appl. No.: 13/880,441

(22) PCT Filed: Oct. 24, 2011
(86) PCT No.: PCT/US11/57415

§ 371 Date: Apr. 19, 2013

Related U.S. Application Data
(60) Provisional application No. 61/407,736, filed on Oct. 28, 2010.

(51) Int. Cl. C07D 413/04; C07D 413/14

(52) U.S. Cl. CPC C07D 413/04; C07D 413/14
USPC 514/212.08; 540/524

(57) ABSTRACT

The present invention is directed to caprolactams which are positive allosteric modulators of metabotropic glutamate receptors, particularly the mGluR5 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamic acid (sometimes referred to simply as L-glutamate or glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception. Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life.

The present invention relates to modulators of metabotropic glutamate receptors, in particular subtype 5 (“mGluR5”) receptors. The mGluR receptors belong to the Type III G-protein coupled receptor (GPCR) superfamily. This superfamily of GPCR's include the calcium-sensing receptors, GABA B receptors and pheromone receptors, which are unique in that they are activated by binding of effectors to the amino-terminus portion of the receptor protein. The mGlu receptors are thought to mediate glutamates demonstrated ability to modulate intracellular signal transduction pathways. They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided into three groups (Groups I, II and III) based on their amino acid sequence homology, their ability to effect certain signal transduction mechanisms, and their known pharmacological properties. Activation of mGluRs lead to a large variety of intracellular responses and activation of different transductional cascades. Among mGluR members, the mGluR5 subtype is of high interest for counterbalancing the deficit or excesses of neurotransmission in neuropsychatric diseases. mGluR5 belongs to Group I and its activation initiates cellular responses through G-protein mediated mechanisms. mGluR5 is coupled to phospholipase C and stimulates phosphoinositide hydrolysis and intracellular calcium mobilization. In the CNS, mGluR5 receptors are abundant mainly throughout cortex, hippocampus, caudate-putamen and nucleus accumbens. As these brain areas have been shown to be involved in emotion, motivational processes and in numerous aspects of cognitive function, mGluR5 modulators are predicted to be of therapeutic interest.

It has become increasingly clear that there is a link between modulation of excitatory amino acid receptors, including the glutamatergic system, through changes in glutamate release or alteration in postsynaptic receptor activation, and a variety of neurological and psychiatric disorders. For example, a variety of potential clinical indications have been suggested to be targets for the development of subtype selective mGluR modulators. These include epilepsy, neuropathic and inflammatory pain, numerous psychiatric disorders (e.g. anxiety and schizophrenia), movement disorders (e.g. Parkinson disease), neuroprotection (stroke and head injury), migraine and addiction/drug dependency. The medical consequences of such glutamate dysfunction make the abatement of these neurological processes an important therapeutic goal.

### SUMMARY OF THE INVENTION

The present invention is directed to caprolactams which are positive allosteric modulators of metabotropic glutamate receptors, particularly the mGluR5 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I:

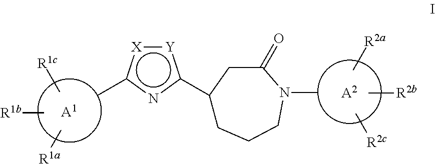

wherein:
A¹ is selected from the group consisting of phenyl, naphthyl and heteroaryl;
A² is selected from the group consisting of phenyl, naphthyl and heteroaryl;
X is selected from N, O and C(R¹³),
Y is selected from N and O,
wherein X is N and Y is O, to form a oxadiazole ring, or
X is O and Y is N, to form a oxadiazole ring, or
X is C(R¹³) and Y is O to form an oxazole ring;
   R^{1a}, R^{1b} and R^{1c} may be absent if the valency of A¹ does not permit such substitution and are independently selected from the group consisting of:
(1) hydrogen,
(2) halogen,
(3) hydroxyl,
(4) —(C═O)ₘ—Oₙ—C₁₋₆alkyl, where m is 0 or 1, n is 0 or 1 (wherein if m is 0 or n is 0, a bond is present) and where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heterocycle, where the heterocycle is unsubstituted or substituted with one or more substituents selected from R¹³,
(10) —(C═O)ₘ—NR¹⁰R¹¹, wherein R¹⁰ and R¹¹ are independently selected from the group consisting of:
   (a) hydrogen,
   (b) C₁₋₆alkyl, which is unsubstituted or substituted with R¹⁴,
   (c) C₃₋₆alkenyl, which is unsubstituted or substituted with R¹⁴,
   (d) C₃₋₆alkynyl, which is unsubstituted or substituted with R¹⁴,
   (e) C₃₋₆cycloalkyl which is unsubstituted or substituted with R¹⁴,
   (f) phenyl, which is unsubstituted or substituted with R¹⁴, and
   (g) heterocycle, which is unsubstituted or substituted with R¹⁴,
(11) —S(O)₂—NR¹⁰R¹¹,
(12) —S(O)_{q}—R¹², where q is 0, 1 or 2 and where R¹² is selected from the definitions of R¹⁰ and R¹¹,
(13) —CO₂H,
(14) —CN, and
(15) —NO₂;
   R^{2a}, R^{2b} and R^{2c} may be absent if the valency of A² does not permit such substitution and are independently selected from the group consisting of:
(1) hydrogen,
(2) halogen,
(3) hydroxyl,
(4) —(C═O)ₘ—Oₙ—C₁₋₆alkyl, where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘC₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heterocycle, where the heterocycle is unsubstituted or substituted with one or more substituents selected from R¹³,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
   R¹³ is selected from the group consisting of:
(1) halogen,
(2) hydroxyl,
(3) —(C═O)ₘ—Oₙ—C₁₋₆alkyl, where the alkyl is unsubstituted or substituted with one or more substituents selected from R¹⁴,
(4) —Oₙ—(C₁₋₃)perfluoroalkyl,
(5) —(C═O)ₘ—Oₙ—C₃₋₆cycloalkyl, where the cycloalkyl is unsubstituted or substituted with one or more substituents selected from R¹⁴,
(6) —(C═O)ₘ—C₂₋₄alkenyl, where the alkenyl is unsubstituted or substituted with one or more substituents selected from R¹⁴,
(7) —(C═O)ₘ—C₂₋₄alkynyl, where the alkynyl is unsubstituted or substituted with one or more substituents selected from R¹⁴,
(8) —(C═O)ₘ—Oₙ-phenyl or —(C═O)ₘ—Oₙ-napthyl, where the phenyl or naphthyl is unsubstituted or substituted with one or more substituents selected from R¹⁴,
(9) —(C═O)ₘ—Oₙ-heterocycle, where the heterocycle is unsubstituted or substituted with one or more substituents selected from R¹⁴,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
   R¹⁴ is selected from the group consisting of:
(1) hydroxyl,
(2) halogen,
(3) C₁₋₆alkyl,
(4) —C₃₋₆cycloalkyl,
(5) —O—C₁₋₆alkyl,
(6) —O(C═O)—C₁₋₆alkyl,
(7) —NH—C₁₋₆alkyl,
(8) phenyl,
(9) heterocycle,
(10) —CO₂H, and
(11) —CN;
   or a pharmaceutically acceptable salt thereof.

An embodiment of the present invention includes compounds of the formula Ia:

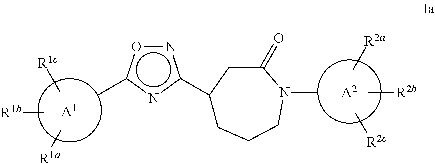

wherein A¹, A², R^{1a}, R^{1b}, R^{1c}, R^{2a}, R^{2b} and R^{2c} are defined herein; or a pharmaceutically acceptable salt thereof.

An embodiment of the present invention includes compounds of the formula Ib:

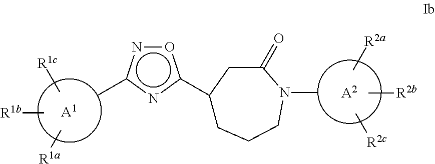

wherein A¹, A², R^{1a}, R^{1b}, R^{1c}, R^{2a}, R^{2b} and R^{2c} are defined herein; or a pharmaceutically acceptable salt thereof.

An embodiment of the present invention includes compounds of the formula Ic:

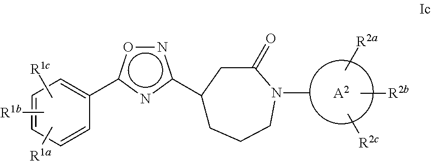

wherein A², R^{1a}, R^{1b}, R^{1c}, R^{2a}, R^{2b} and R^{2c} are defined herein; or a pharmaceutically acceptable salt thereof.

An embodiment of the present invention includes compounds of the formula Id:

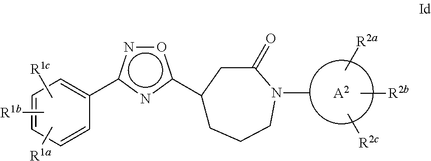

wherein A², R^{1a}, R^{1b}, R^{1c}, R^{2a}, R^{2b} and R^{2c} are defined herein; or a pharmaceutically acceptable salt thereof.

An embodiment of the present invention includes compounds wherein A¹ is selected from the group consisting of phenyl, pyridyl and pyrrolyl. An embodiment of the present invention includes compounds wherein A¹ is phenyl. An embodiment of the present invention includes compounds wherein A¹ is heteroaryl. An embodiment of the present invention includes compounds wherein A¹ is pyridyl. An embodiment of the present invention includes compounds wherein A¹ is pyrrolyl.

An embodiment of the present invention includes compounds wherein A² is selected from the group consisting of: phenyl and pyridyl. An embodiment of the present invention includes compounds where A² is phenyl. An embodiment of the present invention includes compounds wherein A² is heteroaryl. An embodiment of the present invention includes compounds wherein A² is pyridyl.

An embodiment of the present invention includes compounds wherein X is N and Y is O, to form a oxadiazole ring. An embodiment of the present invention includes compounds wherein X is O and Y is N, to form a oxadiazole ring. An embodiment of the present invention includes compounds wherein X is C(R¹³) and Y is O to form an oxazole ring.

An embodiment of the present invention includes compounds wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl, phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

An embodiment of the present invention includes compounds wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl, and
(5) —O—C₁₋₆alkyl, which is unsubstituted or substituted with halogen, hydroxyl or phenyl.

An embodiment of the present invention includes compounds wherein R^{1a}, R^{1b} and R^{1c} are independently selected from the group consisting of:
(1) hydrogen,
(2) halogen, and
(3) C₁₋₆alkyl.

An embodiment of the present invention includes compounds wherein R^{1a}, R^{1b} and R^{1c} are independently selected from the group consisting of
(1) hydrogen,
(2) chloro,
(3) fluroro, and
(4) methyl.

An embodiment of the present invention includes compounds wherein A¹ is phenyl, pyridyl or pyrrolyl and R^{1a}, R^{1b} and R^{1c} are independently selected from the group consisting of:
(1) hydrogen,
(2) chloro,
(3) fluroro, and
(4) methyl.

An embodiment of the present invention includes compounds wherein A¹ is phenyl and wherein R^{1a} is halogen, R^{1b} is hydrogen and R^{1c} is hydrogen. An embodiment of the present invention includes compounds wherein A¹ is phenyl and wherein R^{1a} is fluoro, R^{1b} is hydrogen and R^{1c} is hydrogen. An embodiment of the present invention includes compounds wherein A¹ is phenyl and wherein R^{1a} is chloro, R^{1b} is hydrogen and R^{1c} is hydrogen. An embodiment of the present invention includes compounds wherein A¹ is phenyl and wherein R^{1a} is methyl, R^{1b} is hydrogen and R^{1c} is hydrogen.

An embodiment of the present invention includes compounds wherein A¹ is pyridyl and wherein R^{1a} is halogen, R^{1b} is hydrogen and R^{1c} is hydrogen. An embodiment of the present invention includes compounds wherein A¹ is pyridyl and wherein R^{1a} is fluoro, R^{1b} is hydrogen and R^{1c} is hydrogen. An embodiment of the present invention includes compounds wherein A¹ is pyridyl and wherein R^{1a} is chloro, R^{1b} is hydrogen and R^{1c} is hydrogen. An embodiment of the present invention includes compounds wherein A¹ is pyridyl and wherein R^{1a} is methyl, R^{1b} is hydrogen and R^{1c} is hydrogen.

An embodiment of the present invention includes compounds wherein A¹ is pyrrolyl and wherein R^{1a} is halogen, R^{1b} is hydrogen and R^{1c} is hydrogen. An embodiment of the present invention includes compounds wherein A¹ is pyrrolyl and wherein R^{1a} is fluoro, R^{1b} is hydrogen and R^{1c} is hydrogen. An embodiment of the present invention includes compounds wherein A¹ is pyrrolyl and wherein R^{1a} is chloro, R^{1b} is hydrogen and R^{1c} is hydrogen. An embodiment of the present invention includes compounds wherein A¹ is pyrrolyl and wherein R^{1a} is methyl, R^{1b} is hydrogen and R^{1c} is hydrogen.

An embodiment of the present invention includes compounds wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

An embodiment of the present invention includes compounds wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl,
(5) —O—C₁₋₆alkyl, which is unsubstituted or substituted with halogen, hydroxyl or phenyl, and
(6) —NH—C₁₋₆alkyl, or —N(C₁₋₆alkyl)(C₁₋₆alkyl), which is unsubstituted or substituted with halogen.

An embodiment of the present invention includes compounds wherein R^{2a}, R^{2b} and R^{2c} are independently selected from the group consisting of:
(1) hydrogen,
(2) halogen,
(3) C₁₋₆alkyl, which is unsubstituted or substituted with halogen,
(4) —O—C₁₋₆alkyl, which is unsubstituted or substituted with halogen, and
(5) —NH—C₁₋₆alkyl, or —N(C₁₋₆alkyl)(C₁₋₆alkyl), which is unsubstituted or substituted with halogen.

An embodiment of the present invention includes compounds wherein R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

An embodiment of the present invention includes compounds wherein R^{2a}, R^{2b} and R^{2c} are independently selected from the group consisting of:
(1) hydrogen,
(2) chloro,
(3) fluoro, and
(4) methoxy.

An embodiment of the present invention includes compounds wherein A² is phenyl or pyridyl and R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

An embodiment of the present invention includes compounds wherein A² is phenyl or pyridyl and R^{2a}, R^{2b} and R^{2c} are independently selected from the group consisting of:
(1) hydrogen,
(2) chloro,
(3) fluoro, and
(4) methoxy.

An embodiment of the present invention includes compounds wherein A² is phenyl and wherein R^{2a} is halogen or methoxy, R^{2b} is hydrogen and R^{2c} is hydrogen. An embodiment of the present invention includes compounds wherein A² is phenyl and wherein R^{2a} is fluoro, R^{2b} is hydrogen and R^{2c} is hydrogen. An embodiment of the present invention includes compounds wherein A² is phenyl and wherein R^{2a} is chloro, R^{2b} is hydrogen and R^{2c} is hydrogen. An embodiment of the present invention includes compounds wherein A² is phenyl and wherein R^{2a} is methoxy, R^{2b} is hydrogen and R^{2c} is hydrogen. An embodiment of the present invention includes compounds wherein A² is phenyl and wherein R^{2a} is fluoro, R^{2b} is fluoro and R^{2c} is hydrogen. An embodiment of the present invention includes compounds wherein A² is phenyl and wherein R^{2a} is fluoro, R^{2b} is methoxy and R^{2c} is hydrogen. An embodiment of the present invention includes compounds wherein A² is phenyl and wherein R^{2a} is methoxy, R^{2b} is methoxy and R^{2c} is hydrogen.

An embodiment of the present invention includes compounds wherein A² is pyridyl and wherein R^{2a} is halogen or methoxy, R^{2b} is hydrogen and R^{2c} is hydrogen. An embodiment of the present invention includes compounds wherein A² is pyridyl and wherein R^{2a} is fluoro, R^{2b} is hydrogen and R^{2c} is hydrogen. An embodiment of the present invention includes compounds wherein A² is pyridyl and wherein R^{2a} is chloro, R^{2b} is hydrogen and R^{2c} is hydrogen. An embodiment of the present invention includes compounds wherein A² is pyridyl and wherein R^{2a} is methoxy, R^{2b} is hydrogen and R^{2c} is hydrogen. An embodiment of the present invention includes compounds wherein A² is pyridyl and wherein R^{2a} is fluoro, R^{2b} is fluoro and R^{2c} is hydrogen. An embodiment of the present invention includes compounds wherein A² is pyridyl and wherein R^{2a} is fluoro, R^{2b} is methoxy and R^{2c} is hydrogen. An embodiment of the present invention includes compounds wherein A² is pyridyl and wherein R^{2a} is methoxy, R^{2b} is methoxy and R^{2c} is hydrogen.

An embodiment of the present invention includes compounds wherein is R¹³ hydrogen.

Specific embodiments of the present invention include a compound which is selected from the group consisting of the subject compounds of the Examples herein and pharmaceutically acceptable salts thereof and individual enantiomers and diastereomers thereof.

As appreciated by those of skill in the art, halogen or halo as used herein are intended to include luorine, chlorine, bromine and iodine. Similarly, “alkyl”, as well as other groups having the prefix “alk”, such as alkoxy, alkanoyl, means carbon chains which may be linear or branched or combinations thereof. C₁₋₆, as in C₁₋₆alkyl is defined to identify the group as having 1, 2, 3, 4, 5 or 6 carbons in a linear or branched arrangement, such as methyl, ethyl, n-propyl, iso-propyl, n-butyl, iso-butyl, sec-butyl, tert-butyl, pentyl, hexyl, and the like. “Alkylene” means a straight or branched chain of carbon atoms with a group substituted at both ends, such as —CH₂CH₂— and —CH₂CH₂CH₂—. “Alkenyl” means carbon chains which contain at least one carbon-carbon double bond, and which may be linear or branched or combinations thereof such that C₂₋₆alkenyl is defined to identify the group as having 2, 3, 4, 5 or 6 carbons which incorporates at least one double bond, which may be in a E- or a Z-arrangement, including vinyl, allyl, isopropenyl, pentenyl, hexenyl, heptenyl, 1-propenyl, 2-butenyl, 2-methyl-2-butenyl, and the like. “Alkynyl” means carbon chains which contain at least one carbon-carbon triple bond, and which may be linear or branched or combinations thereof, such as ethynyl, propargyl, 3-methyl-1-pentynyl, 2-heptynyl and the like. “Cycloalkyl” means mono-, bi- or tri-cyclic structures, optionally combined with linear or branched structures, having the indicated number of carbon atoms, such as cyclopropyl, cyclopentyl, cycloheptyl, adamantyl, cyclododecylmethyl, 2-ethyl-1-bicyclo[4.4.0]decyl, and the like. “Alkoxy” means alkoxy groups of a straight or branched having the indicated number of carbon atoms. C₁₋₆alkoxy, for example, includes methoxy, ethoxy, propoxy, isopropoxy, and the like. “Heteroaryl” means mono- or bicyclic aromatic rings with at least one ring containing a heteroatom selected from N, O and S, and each ring containing 5 or 6 atoms. Examples of heteroaryl include benzoimidazolyl, benzimidazolonyl, benzofuranyl, benzofurazanyl, benzopyrazolyl, benzothiazolyl, benzotriazolyl, benzothiophenyl, benzoxazepin, benzoxazolyl, carbazolyl, carbolinyl, cinnolinyl, furanyl, furo(2,3-b)pyridyl, imidazolyl, indolinyl, indolyl, dihydroindolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydroquinoxalinyl, tetrazolyl, tetrazolopyridyl, thiadiazolyl, thiazolyl, thienyl, triazolyl, and N-oxides thereof, and the like.

A group which is designated as being independently substituted with substituents may be independently substituted with multiple numbers of such substituents.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature. Examples of isotopes suitable for inclusion in the compounds of the invention include isotopes of hydrogen such as 2H and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ²³I and ¹²⁵I, and chlorine such as ³⁶Cl. Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula I can generally be prepared by conventional techniques known to those skilled in the art or by processes analogous to those described in the accompanying Examples using appropriate isotopically-labelled reagents in place of the non-labelled reagent previously employed.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particular embodiments include the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particular embodiments citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

Exemplifying the invention are the specific compounds disclosed in the Examples and herein. The subject compounds are useful in a method of enhancing the neuromodulatory effect of metabotorpic glutamate receptor activity in a patient such as a mammal in need of such enhancement comprising the administration of an effective amount of the compound. The present invention is directed to the use of the subject compounds disclosed herein as positive allosteric modulators of metabotropic glutamate receptor activity.

The invention also encompasses a pharmaceutical composition comprising a compound of Formula I in combination with a pharmaceutically acceptable carrier.

The invention also encompasses a method for treating a neurological or psychiatric disorder associated with glutamate dysfunction in a patient in need thereof comprising administering to the patient a therapeutically effective amount of a compound of Formula I. The invention also encompasses this method wherein the neurological or psychiatric disorder associated with glutamate dysfunction is schizophrenia.

The compounds of the present invention are modulators of metabotropic glutamate (mGluR) receptor function, in particular they are positive allosteric modulators of mGluR5 receptors. That is, the compounds of Formula I do not appear to bind to the orthosteric glutamate recognition site, and do not activate the mGluR5 by themselves. Instead, the response of mGluR5 to a concentration of glutamate or mGluR5 agonist is increased when a compound of Formula I is present. The compounds of Formula I are expected to have their effect at mGluR5 by virtue of their ability to enhance the function of the receptor. It is recognized that the compounds of the present invention would be expected to increase the effectiveness of glutamate and glutamate agonists of the mGluR5 receptor. Thus, the compounds of the present invention are expected to be useful in the treatment of various neurological and psychiatric disorders associated with glutamate dysfunction described to be treated herein and others that can be treated by such positive allosteric modulators as are appreciated by those skilled in the art.

The present invention is directed to the use of the compounds disclosed herein as positive allosteric modulators of mGluR5 receptor activity. The present invention is directed to a compound of the present invention or a pharmaceutically acceptable salt thereof for use in medicine. The present invention is further directed to a use of a compound of the present invention or a pharmaceutically acceptable salt thereof for the manufacture of a medicament for positive allosteric modulation of mGluR5 receptor activity or treating the disorders and diseases noted herein in humans and animals.

The present invention is further directed to a method for the manufacture of a medicament for positive allosteric modulation of metabotropic glutamate receptor activity in humans and animals comprising combining a compound of the present invention with a pharmaceutical carrier or diluent.

The subject treated in the present methods is generally a mammal, preferably a human being, male or female, in whom potentiation of metabotropic glutamate receptor activity is desired. In addition to primates, especially humans, a variety of other mammals can be treated according to the method of the present invention. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, eomplexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as positive allosteric modulators of metabotropic glutamate receptor activity, in particular mGluR5 activity, may be readily determined without undue experimentation by methodology well known in the art, including O'Brien et al., Molecular Pharmacology 2003, 64(3) 731-740. In particular, the compounds of the following examples had activity in reference assays by enhancing mGluR5 activity. The utility of the compounds as modulators of metabotropic glutamate receptor 5 (mGluR5) activation was demonstrated by their ability to increase an intracellular calcium flux above that achieved by a sub-threshold level of natural agonist (glutamate). Changes in intracellular Ca²⁺ were measured with Fluo-4AM ester (Invitrogen/Molecular Probes), which was detected on a Fluorometric Imaging Plate Reader (FLIPR, Molecular Devices, Sunnyvale, Calif.). In a typical experiment the mGluR5 positive allosteric modulatory activity of the compounds of the present invention was determined in accordance with the following experimental method.

Cell Culture:

Chinese Hamster Ovary (CHO) cells expressing human mGluR5_{A} were maintained in growth medium containing DMEM, 10% dialyzed Fetal Bovine Serum, 50 units/mL Penicillin, 50 ug/mL Streptomycin, 2 mM L-glutamine, 1×MEM non-essential amino acids, 1 mM sodium pyruvate, 25 mM HEPES, 55 uM 2-mercaptoethanol, 5 ug/mL Puromycin, and 250 ug/mL Zeocin at 37° C. and 5% CO₂. The day before the experiment, the cells were washed and seeded in “plating media” containing only DMEM, 10% dialyzed Fetal Bovine Serum, 50 units/mL Penicillin, and 50 ug/mL Streptomycin at a density of 50,000 cells/well (100 uL/well) in black 384-well clear-bottom PDL-coated plates. The cells were grown overnight at 37° C. and 6% CO₂. This overnight glutamine/glutamate starvation allowed for consistent expression of the mGluR5_{A} receptor, and the ability to add a known amount of agonist (glutamate in most cases) on the day of the experiment.

Fluorescent Ca²⁺ Mobilization (FLIPR) Assay:

The day of the experiment, the cells were washed with 37° C. Assay Buffer (Hanks Balanced Salt Solution with CaCl₂ and MgCl₂, 20 mM HEPES, 2.5 mM Probenecid, 0.1% BSA) with an automated plate washer (3×100 uL, aspiration 3 mm from bottom leaving ˜30 uL of buffer in each well). After washing, 30 uL of dye loading buffer (4 uM Fluo-4AM, 0.04% Pluronic acid, and 1% dialyzed FBS in assay buffer) were added to each well of the plates for 2 uM Fluo-4AM final concentration. The plates were incubated at 37° C. and 6% CO₂ for 1 hour to allow for dye loading. After dye loading, the cells were washed again as above, and placed on the FLIPR. Assays were conducted with two possible scenarios: 1) To determine the potencies of the compounds, as either agonists of mGluR5 or potentiators of mGluR5 in the presence of a sub-threshold amount of glutamate, 10-point titrations of the compounds (1:3 dilution between each point, 30-0.0015 uM final concentrations) were added to the cells, followed by the addition of the EC₂₀ of glutamate (300 nM) to the cells. 2) To determine the cooperativity of the compounds with the natural agonist (glutamate), single concentrations of the compounds were added to the cells, followed by the addition of a 10-point titration of glutamate (1:3 dilution between each point, 1000-0.05 uM final concentrations). When compared to the EC₅₀ of glutamate in the presence of DMSO only on the same assay plate, a left-shift in the glutamate dose-response curve in the presence of compound demonstrates the degree of potentiation at the single concentration of the compound. For both scenarios above, operation of the FLIPR was the same. Baseline fluorescence was monitored for 10 seconds, followed by the addition of compounds diluted in Assay Buffer (1% DMSO concentration after this addition, 0.66% final DMSO concentration after agonist addition). After monitoring fluorescence for 5 minutes, during which time any intrinsic agonist activity of the compounds would have been detected, the agonist (glutamate) also diluted in assay buffer was then added to the cells. The response was then monitored for an additional 3 minutes. In scenario #1, the peak during the final 3 minutes was used for potentiator data, and the peak during the 5 minutes post compound addition was used for compound agonist data. Inflection points for potentiation and agonism were determined with non-linear curve fitting, and the maximal response of the compound was compared to the maximal response of the agonist (1 mM glutamate) to provide a % of max activity for each compound. Additionally, the maximal response of each compound was compared to the sub-threshold response of the agonist (300 nM glutamate) to provide a fold potentiation value at the maximal response.

Potencies for the compounds are reported as EC₅₀ values for agonism (in the absence of 300 nM glutamate) “EC₅₀ values” (actually inflection points) for potentiation (in the presence of 300 nM glutamate).

$$\% Max\_ 1 mM glutamate = \frac{fluorescence counts caused by compound}{fluorescence counts caused by 1 mM glutamate} \times 100 \%$$

$$fold potentiation = \frac{fluorescence counts caused by compound}{fluorescence counts caused by 300 nM glutamate}$$

In scenario #2, the peak during the final 3 minutes was used for the points of the agonist dose response curve. The EC₅₀ values for the agonist in the presence of 0.66% DMSO or each single concentration of the compound were determined with non-linear curve fitting. By dividing the EC₅₀ of glutamate+DMSO by the EC₅₀ of glutamate+compound, the resulting value is the fold-shift in agonist potency, and therefore the degree of potentiation of the compound at the given concentration. This value is called the “glutamate shift”

$$Glutamate shift = \frac{{EC}_{50} of glutamate in the presence of 0.66 \% DMSO}{\begin{matrix}{EC}_{50} of glutamate in the presence \\ of a given concentration of compound\end{matrix}}$$

The compounds of the following Examples were tested and had activity as positive allosteric modulators of the mGluR5 receptor in the foregoing assays. The compounds of the following Examples had activity in potentiating the mGluR5 receptor in the FLIPR assay with an EC₅₀ of about 0.001 μM to 10 μM. In particular, the compounds of Examples 1-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 4-9, 4-10, 4-11, 4-12, 4-13, and 4-14 had activity in potentiating the mGluR5 receptor in the FLIPR assay with an EC₅₀ of about 0.001 μM to 10 μM. For a compound to have thereapeutic utility, it is expected that such compound should have activity in enhancing the mGluR5 receptor in the FLIPR assay with an EC₅₀ of less than about 10 μM.

Metabotropic glutamate receptors including the mGluR5 receptor have been implicated in a wide range of biological functions. This has suggested a potential role for these receptors in a variety of disease processes in humans or other species. See e.g., Byrnes, et al., Neurotherapeutics, 6, 94-107 (2009). The compounds of the present invention have utility in treating, preventing, ameliorating, controlling or reducing the risk of a variety of neurological and psychiatric disorders associated with glutamate dysfunction, including one or more of the following conditions or diseases: schizophrenia or psychosis including schizophrenia (paranoid, disorganized, catatonic, undifferentiated, or residual type), schizophreniform disorder, schizoaffective disorder, for example of the delusional type or the depressive type, delusional disorder, psychotic disorder, brief psychotic disorder, shared psychotic disorder, psychotic disorder due to a general medical condition and substance-induced or drug-induced (for example psychosis induced by alcohol, amphetamine, cannabis, cocaine, hallucinogens, inhalants, opioids, phencyclidine, ketamine and other dissociative anaesthetics, and other psychostimulants), psychosispsychotic disorder, psychosis associated with affective disorders, brief reactive psychosis, schizoaffective psychosis, “schizophrenia-spectrum” disorders such as schizoid or schizotypal personality disorders, personality disorder of the paranoid type, personality disorder of the schizoid type, illness associated with psychosis (such as major depression, manic depressive (bipolar) disorder, Alzheimer's disease and post-traumatic stress syndrome), including both the positive and the negative symptoms of schizophrenia and other psychoses; disorders that comprise as a symptom a deficiency in attention and/or cognition; cognitive disorders including dementia (associated with Alzheimer's disease, ischemia, multi-infarct dementia, trauma, intracranial tumors, cerebral trauma, vascular problems or stroke, alcoholic dementia or other drug-related dementia, AIDS, HIV disease, Parkinson's disease, Huntington's disease, Pick's disease, Creutzfeldt-Jacob disease, perinatal hypoxia, other general medical conditions or substance abuse); Alzheimer's disease, multi-infarct dementia, AIDS-related dementia, and Fronto temperal dementia; delirium, amnestic disorders or age related cognitive decline; migraine, migraine headache; pain including acute pain, chronic pain, severe pain, intractable pain, neuropathic pain, post-traumatic pain, bone and joint pain (osteoarthritis), repetitive motion pain, dental pain, cancer pain, myofascial pain (muscular injury, fibromyalgia), perioperative pain (general surgery, gynecological), chronic pain, neuropathic pain; trigeminal neuralgia; amyotrophic lateral sclerosis (ALS); cerebral deficits subsequent to cardiac bypass surgery and grafting, stroke, cerebral ischemia, spinal cord trauma, head trauma, perinatal hypoxia, cardiac arrest, hypoglycemic neuronal damage; spinal cord injury; neuronal regeneration; neuronal inflammation; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder, drug addiction, tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders, major depressive episode of the mild, moderate or severe type, a manic or mixed mood episode, a hypomanic mood episode, a depressive episode with atypical features, a depressive episode with melancholic features, a depressive episode with catatonic features, a mood episode with postpartum onset, post-stroke depression; major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, for example bipolar I disorder, bipolar II disorder, cyclothymic disorder, depression including unipolar depression, seasonal depression and post-partum depression, premenstrual syndrome (PMS) and premenstrual dysphoric disorder (PDD), mood disorders due to a general medical condition, and substance-induced mood disorders; learning disorders, for example reading disorder, mathematics disorder, or a disorder of written expression, attention-deficit/hyperactivity disorder, and age-related cognitive decline, pervasive developmental disorder including autistic disorder, attention disorders including attention-deficit hyperactivity disorder (ADHD) and conduct disorder; NMDA receptor-related disorders such as autism, depression, benign forgeffulness, childhood learning disorders and closed head injury; neurodegenerative disorders or conditions, neurodegeneration associated with cerebral trauma; neurodegeneration associated with stroke, neurodegeneration associated with cerebral infarct, hypoglycemia-induced neurodegeneration, neurodegeneration associated with epileptic seizure, neurodegeneration associated with neurotoxin poisoning, multi-system atrophy; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Huntington's disease, dyskinesia associated with dopamine agonist therapy, Gilles de la Tourette's syndrome, epilepsy, muscular spasms and disorders associated with muscular spasticity or weakness including tremors; dyskinesias, including tremor (such as rest tremor, postural tremor, intention tremor and essential tremor), tardive dyskinesia, restless leg syndrome, chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), dystonia (including generalised dystonia such as iodiopathie dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Among the disorders above, of particular importance are the treatment of schizophrenia, migraine, anxiety (including agoraphobia, generalized anxiety disorder (GAD), obsessive-compulsive disorder (OCD), panic disorder, posttraumatic stress disorder (PTSD), social phobia, other phobias, substance-induced anxiety disorder), mood disorders (including bipolar disorders (I & II), cyclothymic disorder, depression, dysthymic disorder, major depressive disorder, substance-induced mood disorder), attention-deficit/hyperactivity disorder (ADD, ADHD), eating disorders (Melding anorexia nervosa, bulimia nervosa), epilepsy, cognitive disorders (including delirium, substance-induced persisting delirium, dementia, dementia due to HIV disease, dementia due to Huntington's disease, dementia due to Parkinson's disease, dementia of the Alzheimer's type, substance-induced persisting dementia, mild cognitive impairment), personality disorders (including obsessive-compulsive personality disorder, schizoid, schizotypal disorder), substance-related disorders (including alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence, opioid withdrawal).

In another specific embodiment, the present invention provides a method for treating schizophrenia or psychosis comprising: administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

Thus, in an embodiment the present invention provides a method for treating migraine, comprising: administering to a patient in need thereof an effective amount of a compound of the present invention. In one of the available sources of diagnostic tools, Dorland's Medical Dictionary (23'd Ed., 1982, W. B. Saunders Company, Philidelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another specific embodiment, the present invention provides a method for treating anxiety disorders, comprising: administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another embodiment the present invention provides a method for treating depression, comprising: administering to a patient in need thereof an effective amount of a compound of the present invention. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another embodiment the present invention provides a method for treating epilepsy, comprising: administering to a patient in need thereof an effective amount of a compound of the present invention. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

In a specific embodiment, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating substance-related disorders and addictive behaviors, comprising: administering to a patient in need thereof an effective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another specific embodiment, the present invention provides a method for treating pain, comprising: administering to a patient in need thereof an effective amount of a compound of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The subject compounds are further useful in a method for the prevention, treatment, control, amelioration, or reduction of risk of the diseases, disorders and conditions noted herein. The subject compounds are further useful in a method for the prevention, treatment, control, amelioration, or reduction of risk of the aforementioned diseases, disorders and conditions in combination with other agents. The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention may be desirable. However, the combination therapy may also includes therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. Accordingly, the pharmaceutical compositions of the present invention include those that also contain one or more other active ingredients, in addition to a compound of the present invention. The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, atypical antipsychotics, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexol)hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

The subject compounds are further useful in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 30 mg/kg. of body weight daily are administered to the patient, e.g., humans and elderly humans. The dosage range will generally be about 0.5 mg to 5.0 g. per patient per day which may be administered in single or multiple doses. In one embodiment, the dosage range will be about 0.5 mg to 2.5 mg per patient per day; in another embodiment about 0.5 mg to 1 g per patient per day; in yet another embodiment about 5 mg to 500 mg per patient per day; and in yet another embodiment about 5 mg to 100 mg per patient per day. Pharmaceutical compositions of the present invention may be provided in a solid dosage formulation such as comprising about 0.5 mg to 800 mg active ingredient, or comprising about 1 mg to 400 mg active ingredient. The pharmaceutical composition may be provided in a solid dosage formulation comprising about 1 mg, 5 mg, 10 mg, 25 mg, 50 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, such as once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 5000 milligrams, preferably from about 1 milligrams to about 1000 milligrams. In the case of a 70 kg adult human, the total daily dose will generally be from about 7 milligrams to about 800 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials and the requisite intermediates are in some cases commercially available, or can be prepared according to literature procedures or as illustrated herein. The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures. Starting materials are made according to procedures known in the art or as illustrated herein. The following abbreviations are used herein: Me: methyl; Et: ethyl; t-Bu: tert-butyl; Ar: aryl; Ph: phenyl; Bn: benzyl; Ac: acetyl; THF: tetrahydrofuran; DIEA: N,N-diisopropylethylamine; DMSO: dimethylsulfoxide; EDC: N-(3-dimethylaminopropyl)-N′-ethylcarbodiimide hydrochloride; HOBT: hydroxybenzotriazole hydrate; Boc: tert-butyloxy carbonyl; Et₃N: triethylamine; EtOAc: ethyl acetate; CH₂Cl₂: dichloromethane; CH₃OH: methanol; C₂H₅OH: ethanol; CH₃CN: acetonitrile; BSA: bovine serum albumin; TFA: trifluoracetic acid; DMF: N,N-dimethylformamide; MTBE: methyl tert-butyl ether; SOCl₂: thionyl chloride; CDI: carbonyl diimidazole; RT: room temperature; HPLC: high performance liquid chromatography; TEMPO: 2,2,6,6-tetramethyl-1-piperidine 1-oxyl; HATU: O-(7-azabenzotriazol-1-yl)-N,N,′,′-tetramethyluronium hexafluorophosphate; Burgess reagent: methoxycarbonylsulfamoyl) trimethylammonium inner salt. The compounds of the present invention can be prepared in a variety of fashions.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

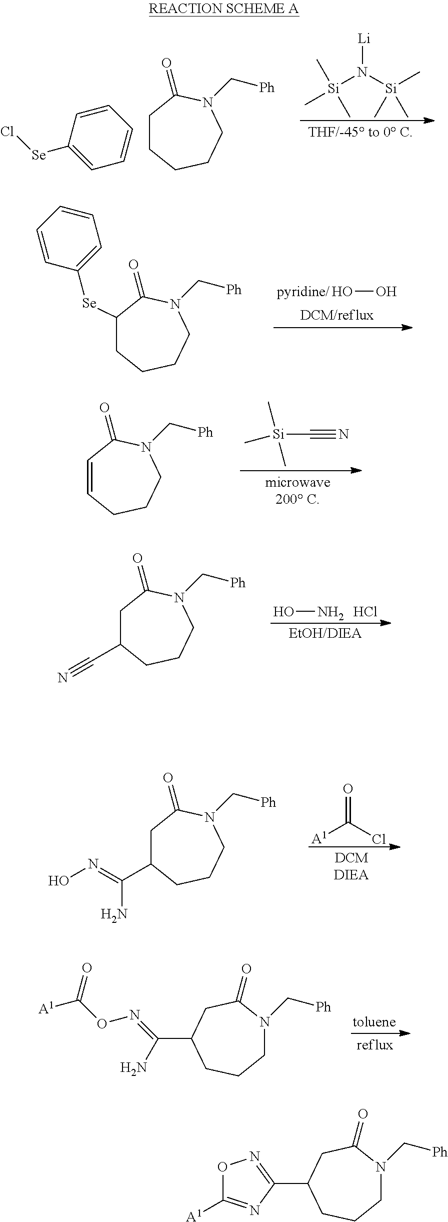

As shown in Reaction Scheme A, reaction of the phenylselenide with the cycylic lactam in the presence of Li TDMS gives the selenoether which is reacted with pyridine in the presence of hydrogen peroxide to give the unsaturated lactone. Treatment with TMS-CN followed by hydroxylamine gives the carboximdamide. Reaction with an aryl acyl halide followed by heating to form the oxadiazole ring gives the benzyl caprolactam.

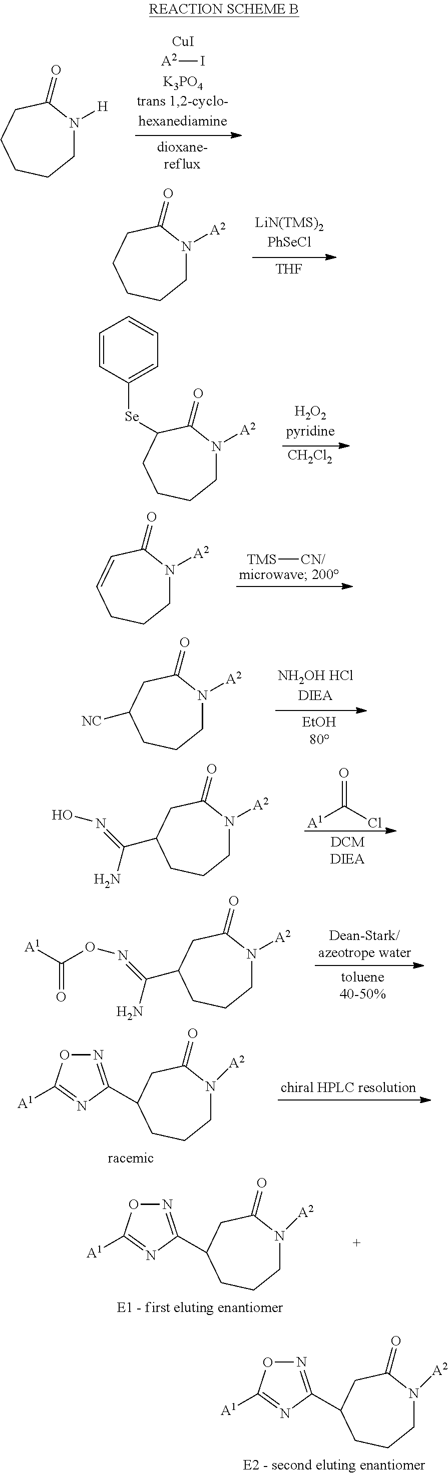

As shown in Reaction Scheme B, acylation of the amide nitrogen with an aryl iodide, followed by reaction of the phenylselenide with the cycylic lactam in the presence of Li TDMS gives the selenoether which is reacted with pyridine in the presence of hydrogen peroxide to give the unsaturated lactone. Treatment with TMS-CN followed by hydroxylamine gives the carboximdamide. Reaction with an aryl acyl halide followed by heating to form the oxadiazole ring gives the aryl caprolactam. Chiral HPLC is employed to separate the individual enantiomers.

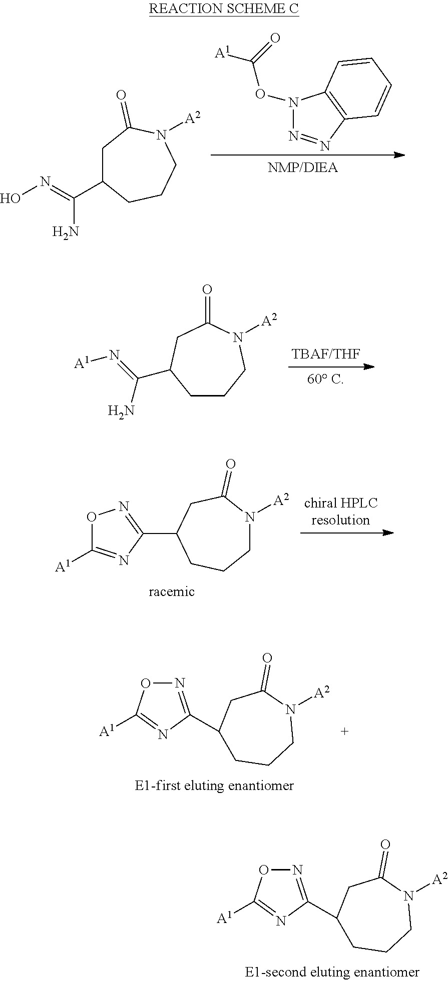

As shown in Reaction Scheme C, the carboximdamide may be reacted with an activated aryl acid followed by reaction with TBAF to form the oxadiazole ring to give the aryl caprolactam. Chiral HPLC is employed to separate the individual enantiomers.

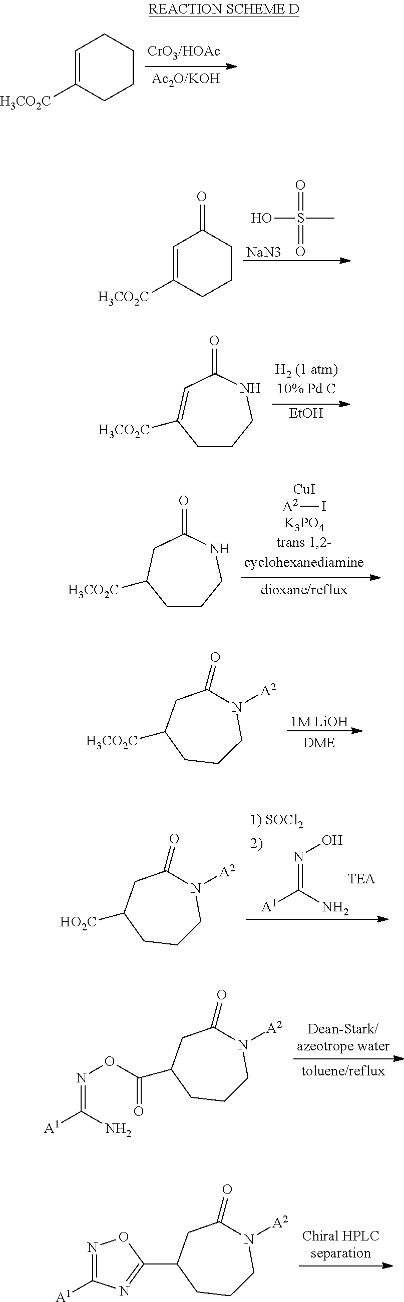

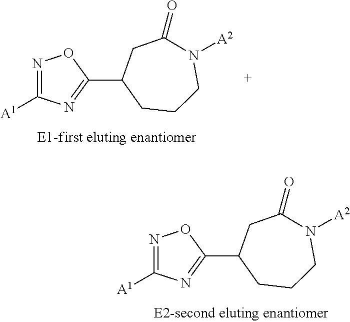

As shown in Reaction Scheme D, ring expansion of the cyclohexene carboxylate gives the substituted caprolactam. Acylation of the amide nitrogen with an aryl iodide, followed by hydrolysis of the ester and reaction with hydroxylamine gives the carboximdamide. Dean-Stark condensation to form the oxadiazole ring gives the aryl caprolactam. Chiral HPLC is employed to separate the individual enantiomers.

### Example 1

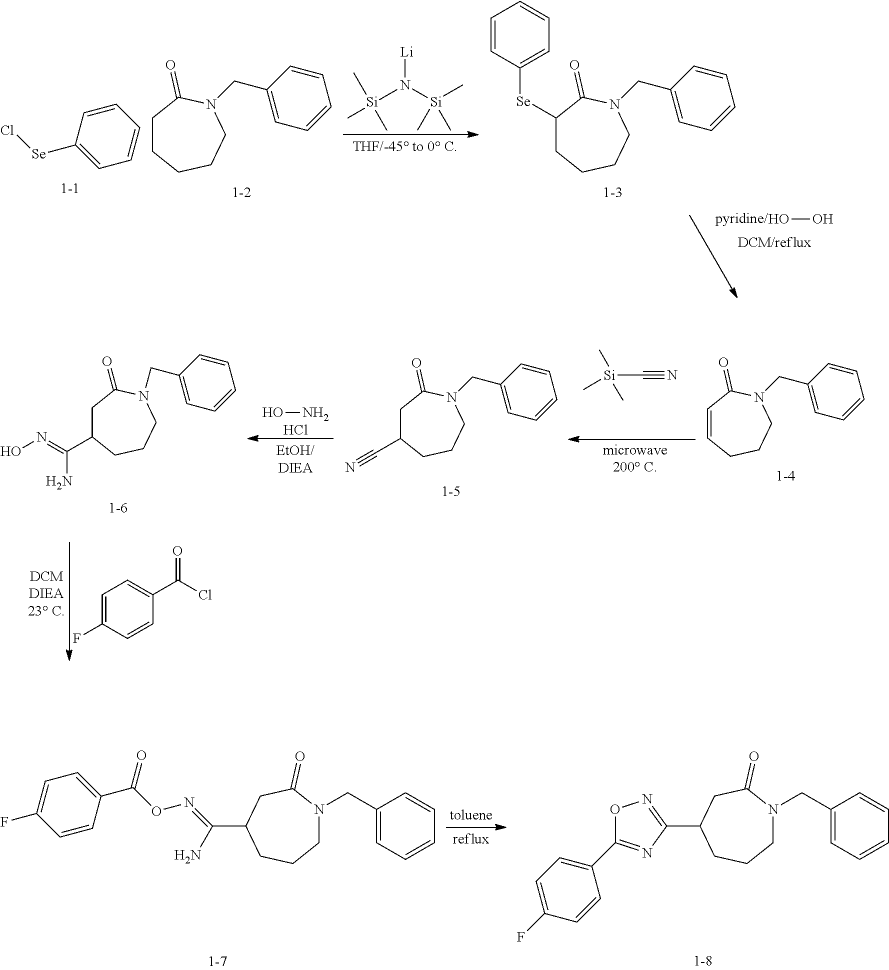

### 1-benzyl-3-(phenylselanyl)azepan-2-one (1-3)

A solution of 1-2 (1.87 g, 9.20 mmol) in anhydrous THF (6.5 ml) under nitrogen was cooled (−45° C.). The solution was treated with lithium bis(trimethylsilyl)amide (18.4 ml, 18.4 mmol) and the solution was warmed to 0° C. for 30 min. Recooled the solution to −45° C. then added a solution of 1-1 (1.85 g, 9.66 mmol) in anhydrous THF (4 ml). The solution was slowly warmed to room temperature over 5 hours. The reaction solution was poured into 1N HCl (8 ml) and the organic layer separated. The aqueous layer was extracted with ethyl acetate (2×8 ml). The organics were combined and washed with brine, dried with magnesium sulfate and then concentrated to yield 1-3 as an oil (3.28 g, 100%). Data for 1-3: LRMS m/z (M+H): 360.4.

### 1-benzyl-1,5,6,7-tetrahydro-2H-azepin-2-one (1-4)

A DCM solution (70 ml) of 1-3 (3.28 g, 9.15 mmol)) was treated with pyridine (1.49 g, 18.31 mmol) followed by hydrogen peroxide (5.34 g, 54.9 mmol). This was heated to reflux. Upon completion the reaction was cooled to ambient temp and the organic layer was separated and washed with dilute sodium bicarbonate (3×40 ml) followed by 1N HCl (3×40 ml) then brine. The organic layer was dried over magnesium sulfate and concentrated to an oil. This was purified by gradient elution on silica gel (0 to 10% methanol in DCM) to yield 1-4 as a dark oil (1.29 g, 70%). Data for 1-4: LRMS m/z (M+H): 202.6.

### 1-benzyl-2-oxoazepane-4-carbonitrile (1-5)

A mixture of 1-4 (725 mg, 3.60 mmol) and TMS-CN (1.07 g, 10.8 mmol) was irradiated for 20 minutes at 200° C. in a Biotage Initiator microwave reactor. The reaction was purified on a Gilson reverse phase hplc to recover 1-5 as an oil (403 mg, 49%). Data for 1-5: LRMS m/z (M+H): 229.3.

### 1-benzyl-N′-hydroxy-2-oxoazepane-4-carboximidamide (1-6)

Ethanol (2 ml) and hydroxylamine hydrochloride (100 mg, 1.43 mmol) were added to a sealed tube. DIEA (222 mg, 1.72 mmol) was then added in one portion with stirring at room temperature for 15 minutes. 1-5 (218 mg, 0.955 mmol) was added as an ethanol (1 ml) solution. This mixture was then heated to 80° C. in a sealed reaction tube. Upon completion the reaction was evaporated and used crude in the next step. Data for 1-6: LRMS m/z (M+H): 262.5.

### 1-benzyl-N′-{[(4-fluorophenyl)carbonyl]oxy}-2-oxoazepane-4-carboximidamide (1-7)

Crude 1-6 (250 mg, 0.96 mmol) was dissolved in DCM (2 ml) then DIEA (371 mg, 2.87 mmol) was added followed by a DCM solution (2 ml) of 4-fluorobenzoyl chloride (228 mg, 1.44 mmol). The reaction was stirred at ambient temperature. The crude reaction was purified by gradient elution on silica gel (0 to 5% methanol in DCM) to yield solid 1-7 (260 mg, 71%). Data for 1-7: LRMS m/z (M+H): 384.4.

### 1-benzyl-4-[5-(4-fluorophenyl)-1,2,4-oxadiazol-3-yl]azepan-2-one (1-8)

In a 50 mL RBF equipped with a stir bar, a Dean-Stark trap, toluene (20 ml) and a thermocouple was added 1-8 (260 mg, 0.68 mmol). The oil bath was heated to 132° C. removing the toluene-water azeotrope over 6 hours. The reaction was then cooled to room temp and dilute sodium bicarbonate (25 ml) was added along with ethyl acetate (25 ml). The aqueous was drawn off and the organic was dried with magnesium sulfate, filtered and evaporated to yield an oil. The crude material was purified by gradient elution on a Gilson reverse phase hplc to yield solid 1-8 (160 mg, 64%). Data for 1-8: LRMS m/z (M+H): 366.4. 400 Mhz H¹ NMR (CDCl₃): δ8.14 (m, 2H), 7.30 (m, 5H), 7.21 (m, 2H), 4.65 (q, 2H), 3.53 (m, 1H), 3.35 (m, 1H), 3.26 (m, 1H), 3.10 (m, 2H), 2.25 (m, 1H), 1.91 (m, 2H), 1.49 (m, 1H).

### Example 2

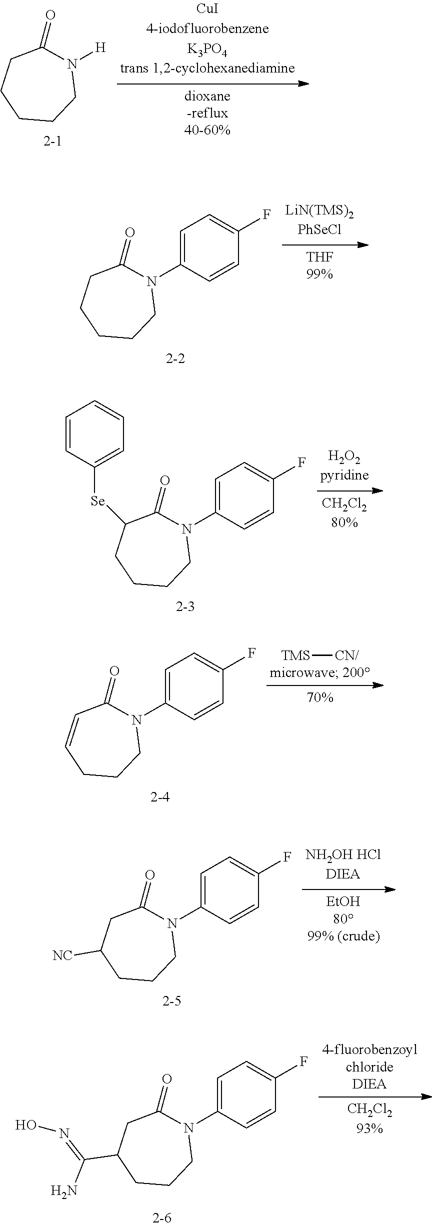

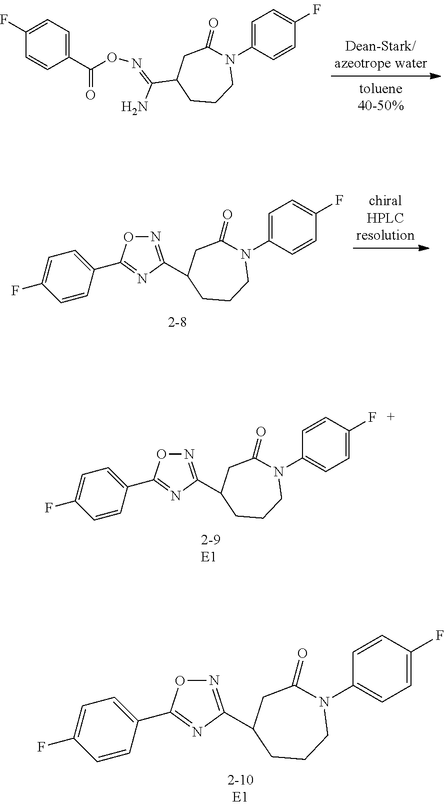

### 1-(4-fluorophenyl)azepan-2-one (2-2)

2-1 (1.3 g, 11.7 mmol), 4-iodofluorobenzene (2.0 g, 9.0 mmol), copper iodide (0.051 g, 0.27 mmol), trans-1,2-cyclohexanediamine (0.33 g, 2.88 mmol) and potassium phosphate tribasic (5.6 g, 21.6 mmol) were suspended in anhydrous dioxane and refluxed overnight. The reaction was cooled, diluted with dioxane and then filtered through a short plug of silica. The filter cake was washed with ethyl acetate. The filtrate was then washed with dilute HCl, and evaporated to an oil. The oil was purified by gradient elution on silica gel (0 to 10% methanol in DCM) to yield 2-2 as a dark oil (1.19 g, 64%). Data for 2-2: LRMS m/z (M+H): 208.5.

### 1-(4-fluorophenyl)-3-(phenylselanyl)azepan-2-one (2-3)

Crude 2-3 was prepared in a manner analogous to 1-3 above. Pure 2-2 (1.19 g, 5.74 mmol), lithium bis(trimethylsilyl)amide (11.5 ml, 11.5 mmol) and phenylselenyl chloride (1.16 g, 6.03 mmol) were used to yield crude 2-3 as a dark oil (2.18 g, 100%). Data for 2-3: LRMS m/z (M+H): 364.3.

### 1-(4-fluorophenyl)-1,5,6,7-tetrahydro-2H-azepin-2-one (2-4)

Crude 2-4 was prepared in a manner analogous to 1-4 above. Crude 2-3 (2.18 g, 6.02 mmol), pyridine (0.95 g, 12.3 mmol) and hydrogen peroxide (3.51 g, 36.1 mmol) were used to yield 2-4 as a dark oil (905 mg, 73%). Data for 2-4: LRMS m/z (M+H): 206.4.

### 1-(4-fluorophenyl)-2-oxoazepane-4-carbonitrile (2-5)

2-5 was prepared in a manner analogous to 1-5 above. Crude 2-4 (905 mg, 4.41 mmol) and TMS-CN (875 g, 8.82 mmol) were irradiated for 20 minutes at 200° C. in a Biotage Initiator microwave reactor. The reaction was purified by gradient elution on silica gel (0 to 10% methanol in DCM) to yield 2-5 as a dark oil (725 mg, 70%). Data for 2-5: LRMS m/z (M+H): 233.4.

### 1-(4-fluorophenyl)-N′-hydroxy-2-oxoazepane-4-carboximidamide (2-6)

2-6 was prepared in a manner analogous to 1-6 above. Ethanol (4 ml), hydroxylamine hydrochloride (110 mg, 1.58 mmol), DIEA (245 mg, 1.90 mmol) and 2-5 (245 mg, 1.06 mmol) were heated to 80° C. in a sealed reaction tube. Upon completion the reaction was evaporated and used crude in the next step. Data for 2-6: LRMS m/z (M+H): 266.4.

### 1-(4-fluorophenyl)-N′-{[(4-fluorophenyl)carbonyl]oxy}-2-oxoazepane-4-carboximidamide (2-7)

2-7 was prepared in a manner analogous to 1-7 above using 2-6 (280 mg, 1.06 mmol), 4-fluorobenzoyl chloride (251 mg, 1.58 mmol) and DIEA (409 mg, 3.17 mmol) in DCM. The crude reaction was purified by gradient elution on silica gel (0 to 10% methanol in DCM) to yield 2-7 (380 mg, 93%). Data for 2-7: LRMS m/z (M+H): 388.3.

### 1-(4-fluorophenyl)-4-[5-(4-fluorophenyl)-1,2,4-oxadiazol-3-yl]azepan-2-one (2-8, racemate)

2-8 was prepared in a manner analogous to 1-8 above from 2-7 (380 mg, 0.981 mmol). The crude material was purified on a Gilson reverse phase hplc to yield solid 2-8 (67 mg, 19%) as a racemate. Data for 2-8: HRMS m/z (M+H): 370.1369 found. This racemate was separated into the two enantiomers on a chiral AS-H column.

### 1-(4-fluorophenyl)-4-[5-(4-fluorophenyl)-1,2,4-oxadiazol-3-yl]azepan-2-one (2-9, E1)

This compound was the first eluting isomer off the chiral column. Data for 2-9: HRMS m/z (M+H): 370.1369. 400 Mhz H¹ NMR (CDCl₃): δ8.14 (m, 2H), 7.23 (m, 4H), 7.08 (t, 2H), 3.98 (m, 1H), 3.75 (dd, 1H), 3.43 (m, 1H), 3.19, (m, 2H), 2.40 (m, 1H), 2.12 (m, 2H), 1.92 (m, 1H).

### 1-(4-fluorophenyl)-4-[5-(4-fluorophenyl)-1,2,4-oxadiazol-3-yl]azepan-2-one (2-10, E2)

This compound was the second eluting isomer off the chiral column. Data for 2-10: HRMS m/z (M+H): 370.1369. 400 Mhz H¹ NMR (CDCl₃): δ8.14 (m, 2H), 7.23 (m, 4H), 7.08 (t, 2H), 3.98 (m, 1H), 3.75 (dd, 1H), 3.43 (m, 1H), 3.19, (m, 2H), 2.40 (m, 1H), 2.13 (m, 2H), 1.93 (m, 1H).

The following compounds were prepared using the foregoing methodology, but substituting the appropriately substituted reagent, as described in the foregoing Reaction Schemes and Examples. The requisite starting materials were commercially available, described in the literature or readily synthesized by one skilled in the art of organic synthesis without undue experimentation. Enantiomers are labelled as E1 (first eluting) and E2 (second eluting) after separation by chiral chromatography.

| Cpd | Structure | Name | HRMS m/z (M + 1) |
|---|---|---|---|
| 2-10 | 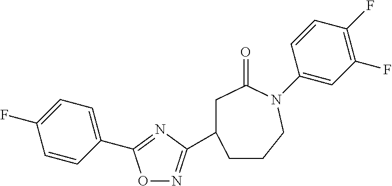 E1 | 1-(3,4-difluorophenyl)- 4-[5-(4-fluorophenyl)- 1,2,4-oxadiazol-3- yl]azepan-2-one | 388.1264 found 388.1267 required |
| 2-11 | 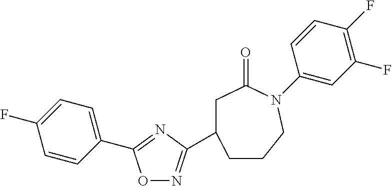 E2 | 1-(3,4-difluorophenyl)- 4-[5-(4-fluorophenyl)- 1,2,4-oxadiazol-3- yl]azepan-2-one | 388.1 found 388.1 required |
| 2-12 | 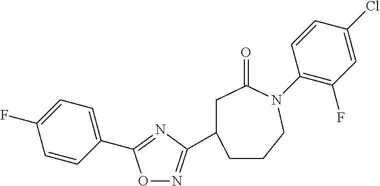 E1 | 1-(4-chloro-2- fluorophenyl)-4-[5-(4- fluorophenyl)-1,2,4- oxadiazol-3-yl]azepan- 2-one | 404.0972 found 404.0972 required |
| 2-13 | 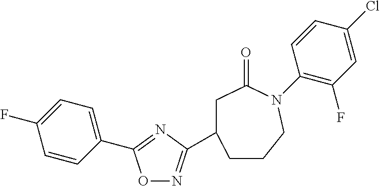 E2 | 1-(4-chloro-2- fluorophenyl)-4-[5-(4- fluorophenyl)-1,2,4- oxadiazol-3-yl]azepan- 2-one | 404.1 found 404.1 required |
| 2-14 | 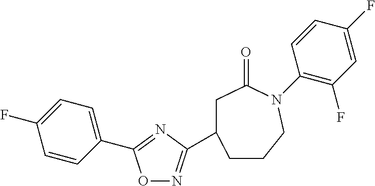 E1 | 1-(2,4-difluorophenyl)- 4-[5-(4-fluorophenyl)- 1,2,4-oxadiazol-3- yl]azepan-2-one | 388.1272 found 388.1267 required |
| 2-15 | 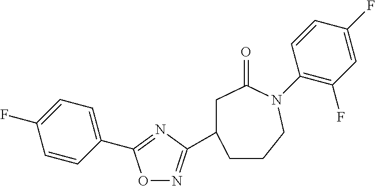 E2 | 1-(2,4-difluorophenyl)- 4-[5-(4-fluorophenyl)- 1,2,4-oxadiazol-3- yl]azepan-2-one | 387.9 found 388.1 required |
| 2-16 | E1 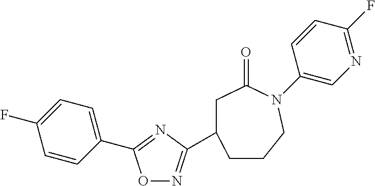 | 4-[5-(4-fluorophenyl)- 1,2,4-oxadiazol-3-yl]-1- (6-fluoropyridin-3- yl)azepan-2-one | 371.1321 found 371.1314 required |
| 2-17 | 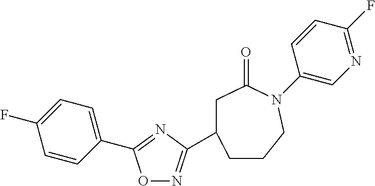 E2 | 4-[5-(4-fluorophenyl)- 1,2,4-oxadiazol-3-yl]-1- (6-fluoropyridin-3- yl)azepan-2-one | 371.0 found 371.1 required |
| 2-18 | 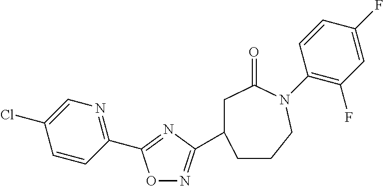 E1 | 4-[5-(5-chloropyridin-2- yl)-1,2,4-oxadiazol-3- yl)-1-(2,4- difluorophenyl)azepan- 2-one | 405.0934 found 405.0924 required |
| 2-19 | E2 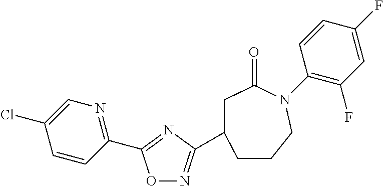 | 4-[5-(5-chloropyridin-2- yl)-1,2,4-oxadiazol-3- yl]-1-(2,4- difluorophenyl)azepan- 2-one | 404.9 found 405.0 required |
| 2-20 | 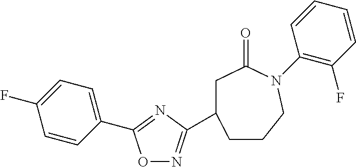 E1 | 1-(2-fluorophenyl)-4-[5- (4-fluorophenyl)-1,2,4- oxadiazol-3-yl]azepan- 2-one | 370.1365 found 370.1362 required |
| 2-21 | 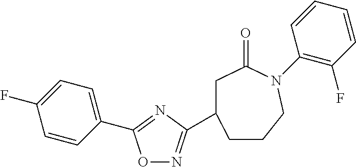 E2 | 1-(2-fluorophenyl)-4-[5- (4-fluorophenyl)-1,2,4- oxadiazol-3-yl]azepan- 2-one | 369.9 found 370.1 required |
| 2-22 | 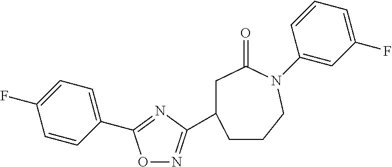 E1 | 1-(3-fluorophenyl)-4-[5- (4-fluorophenyl)-1,2,4- oxadiazol-3-yl]azepan- 2-one | 370.1366 found 370.1362 required |
| 2-23 | 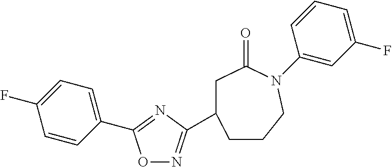 E2 | 1-(3-fluorophenyl)-4-[5- (4-fluorophenyl)-1,2,4- oxadiazol-3-yl]azepan- 2-one | 370.1 found 370.1 required |
| 2-25 | 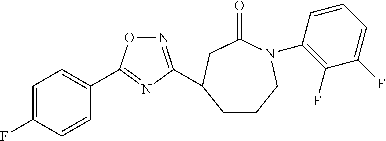 E1 | 1-(2,3-difluorophenyl)- 4-[5-(4-fluorophenyl)- 1,2,4-oxadiazol-3- yl]azepan-2-one | 388.1261 found 388.1267 required |
| 2-26 | 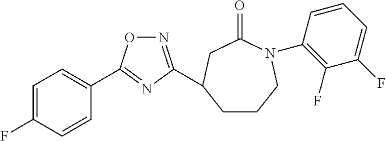 E2 | 1-(2,3-difluorophenyl)- 4-[5-(4-fluorophenyl)- 1,2,4-oxadiazol-3- yl]azepan-2-one | 388.1262 found 388.1267 required |
| 2-27 | 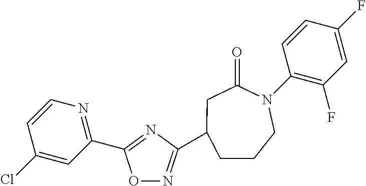 Racemic | 4-[5-(4-chloropyridin-2- yl)-1,2,4-oxadiazol-3- yl]-1-(2,4- difluorophenyl)azepan- 2-one | 405.0922 found 405.0924 required |
| 2-28 | 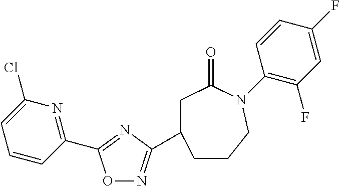 Racemic | 4-[5-(6-chloropyridin-2- yl)-1,2,4-oxadiazol-3- yl]-1-(2,4- difluorophenyl)azepan- 2-one | 404.9 found 405.0 required |

### Example 3

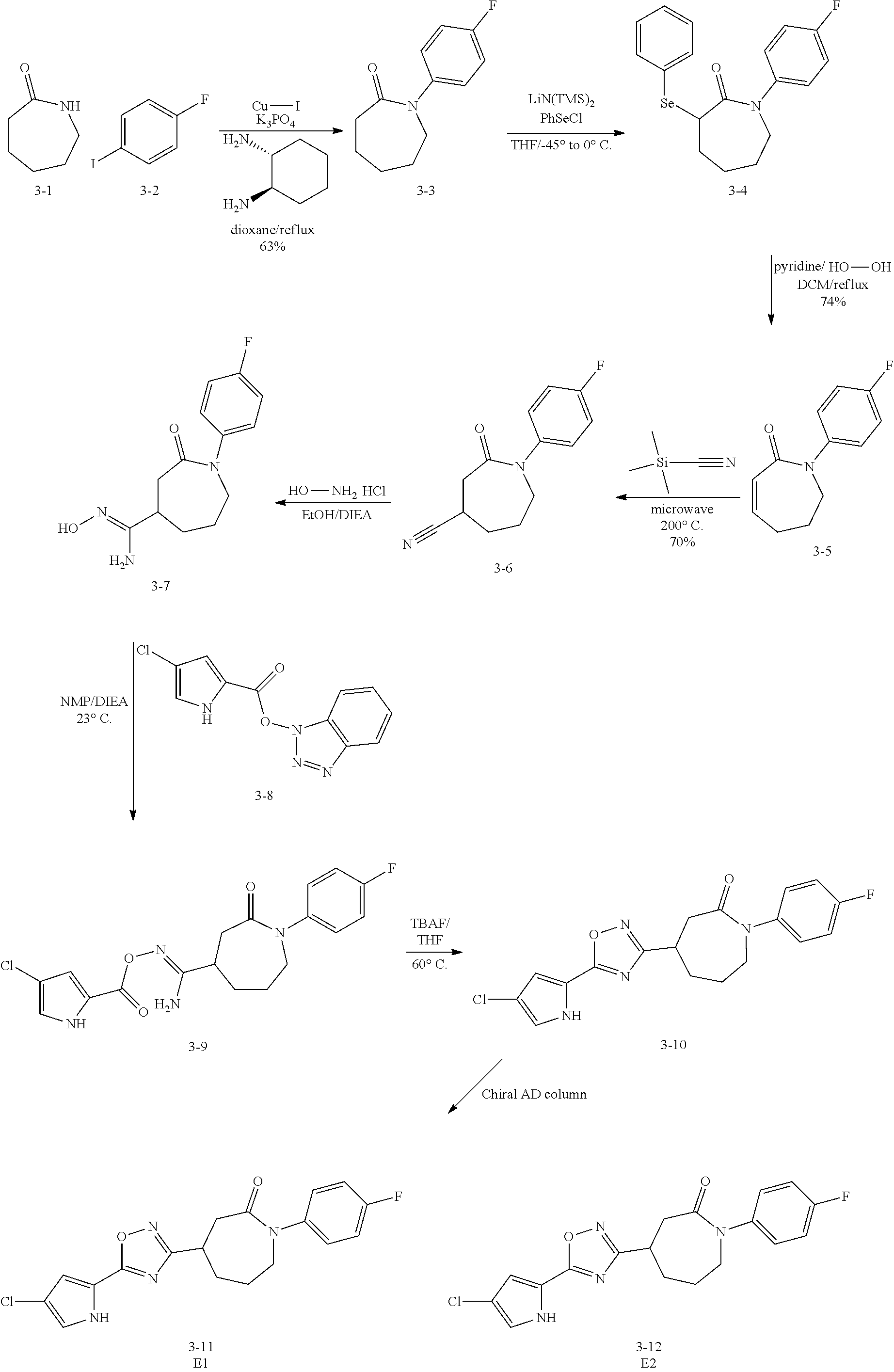

### 1-(4-fluorophenyl)azepan-2-one (3-3)

3-1 (1.3 g, 11.7 mmol), 3-2 (2.0 g, 9.0 mmol), copper iodide (0.051 g, 0.27 mmol), trans-1,2-cyclohexanediamine (0.33 g, 2.88 mmol) and potassium phosphate tribasic (5.6 g, 21.6 mmol) were suspended in anhydrous dioxane and refluxed overnight. The reaction was cooled, diluted with dioxane and then filtered through a short plug of silica. The filter cake was washed with ethyl acetate. The filtrate was then washed with dilute HCl, and evaporated to an oil. The oil was purified by gradient elution on silica gel (0 to 10% methanol in DCM) to yield 3-3 as a dark oil (1.19 g, 64%). Data for 3-3: LRMS m/z (M+H): 208.5.

### 1-(4-fluorophenyl)-3-(phenylselanyl)azepan-2-one (3-4)

Crude 3-4 was prepared in a manner analogous to 1-3 above. Pure 3-3 (1.19 g, 5.74 mmol), lithium bis(trimethylsilyl)amide (11.5 ml, 11.5 mmol) and phenylselanyl chloride (1.16 g, 6.03 mmol) were used to yield crude 3-4 as a dark oil (2.18 g, 100%). Data for 3-4: LRMS m/z (M+H): 364.3.

### 1-(4-fluorophenyl)-1,5,6,7-tetrahydro-2H-azepin-2-one (3-5)

Crude 3-5 was prepared in a manner analogous to 1-4 above. Crude 3-4 (2.18 g, 6.02 mmol), pyridine (0.95 g, 12.3 mmol) and hydrogen peroxide (3.51 g, 36.1 mmol) were used to yield 3-5 as a dark oil (905 mg, 73%). Data for 3-5: LRMS m/z (M+H): 206.4.

### 1-(4-fluorophenyl)-2-oxoazepane-4-carbonitrile (3-6)

3-6 was prepared in a manner analogous to 1-5 above. Crude 3-5 (905 mg, 4.41 mmol) and TMS-CN (875 g, 8.82 mmol) were irradiated for 20 minutes at 200° C. in a Biotage Initiator microwave reactor. The reaction was purified by gradient elution on silica gel (0 to 10% methanol in DCM) to yield 3-6 as a dark oil (725 mg, 70%). Data for 3-6: LRMS m/z (M+H): 233.4.

### 1-(4-fluorophenyl)-N′-hydroxy-2-oxoazepane-4-carboximidamide (3-7)

3-7 was prepared in a manner analogous to 1-6 above. Ethanol (4 ml), hydroxylamine hydrochloride (110 mg, 1.58 mmol), DIEA (245 mg, 1.90 mmol) and 3-6 (245 mg, 1.06 mmol) were heated to 80° C. in a sealed reaction tube. Upon completion the reaction was evaporated and used crude in the next step. Data for 3-7: LRMS m/z (M+H): 266.4.

### N′-{[(4-chloro-1H-pyrrol-2-yl)carbonyl]oxy}-1-(4-fluorophenyl)-2-oxoazepane-4-carboximidamide (3-9)

To an NMP solution (7 ml) of crude 3-7 (280 mg, 1.06 mmol) was added 3-8 (310 mg, 1.18 mmol) and DIEA (208 mg, 1.61 mmol). This was stirred at ambient temperature until complete. The reaction was diluted with ethyl acetate (50 ml), washed with dilute sodium bicarbonate (2×25 ml) then the organic layer was drawn off, dried with magnesium sulfate and evaporated. This solid was recrystallized from warm DCM/MeOH to yield 3-9 (177 mg, 42%). Data for 3-9: LRMS m/z (M+H): 393.3.

### 4-[5-(4-chloro-1H-pyrrol-2-yl)-1,2,4-oxadiazol-3-yl]-1-(4-fluorophenyl)azepan-2-one (3-10)

3-9 (212 mg, 0.54 mmol) was suspended in dry THF under nitrogen. To this was added TBAF (423 mg, 1.62 mmol) which resulted in the immediate dissolution of the solid. The reaction was heated at 60° C. until complete. The solution was evaporated to an oil and was purified by gradient elution on a Gilson reverse phase hplc to yield solid 3-10 (125 mg, 61%). Data for 3-10: LRMS m/z (M+H): 375.3. This racemate was separated into the two enantiomers on a chiral AD column.

### 4-[5-(4-chloro-1H-pyrrol-2-yl)-1,2,4-oxadiazol-3-yl]-1-(4-fluorophenyl)azepan-2-one (3-11, E1)

This compound was the first eluting isomer off the chiral AD column. Data for 3-11: HRMS m/z (M+H): 375.1023. 400 Mhz H¹NMR (DMSO-d₆): δ7.30 (m, 3H), 7.21 (m, 2H), 7.03 (s, 1H), 3.96 (m, 1H), 3.65 (dd, 1H), 3.30 (m, 1H), 3.16 (m, 1H), 2.86 (m, 1H), 2.23 (m, 1H), 2.00 (m, 1H), 1.85 (m, 1H).

### 4-[5-(4-chloro-1H-pyrrol-2-yl)-1,2,4-oxadiazol-3-yl]-1-(4-fluorophenyl)azepan-2-one (3-12, E2)

This compound was the second eluting isomer off the chiral AD column. Data for 3-12: HRMS m/z (M+H): 375.1023. 400 Mhz H¹ NMR (DMSO-d₆): δ 7.28 (m, 3H), 7.21 (m, 2H), 7.03 (s, 1H), 3.96 (q, 1H), 3.63 (dd, 1H), 3.30 (m, 1H), 3.16 (m, 1H), 2.86 (m, 1H), 2.24 (m, 1H), 2.00 (m, 1H), 1.85 (m, 1H).

The following compounds were prepared using the foregoing methodology, but substituting the appropriately substituted reagent, as described in the foregoing Reaction Schemes and Examples. The requisite starting materials were commercially available, described in the literature or readily synthesized by one skilled in the art of organic synthesis without undue experimentation.

| Cpd | Structure | Name | HRMS m/z (M + 1) |
|---|---|---|---|
| 3-13 | E1 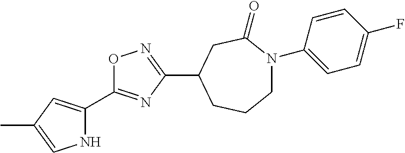 | 1-(4-fluorophenyl)-4- [5-(4-methyl-1H-pyrrol- 2-yl)-1,2,4-oxadiazol-3- yl]azepan-2-one | 355.1569 found 354.1492 required |
| 3-14 | 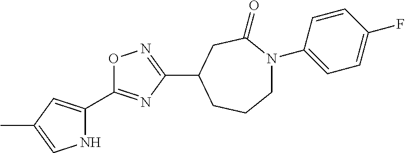 E2 | 1-(4-fluorophenyl)-4- [5-(4-methyl-1H-pyrrol- 2-yl)-1,2,4-oxadiazol-3- yl]azepan-2-one | 355.1569 found 354.1492 required |
| 3-15 | 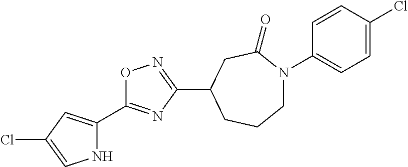 Racemic | 1-(4-chlorophenyl)-4- [5-(4-chloro-1H-pyrrol- 2-yl)-1,2,4-oxadiazol-3- yl]azepan-2-one | 391.0724 found 390.0650 required |
| 3-16 | 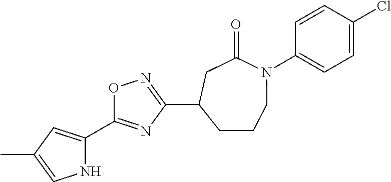 Racemic | 1-(4-chlorophenyl)-4- [5-(4-methyl-1H-pyrrol- 2-yl)-1,2,4-oxadiazol-3- yl]azepan-2-one | 371.1275 found 370.1197 required Not resolved |
| 3-17 | 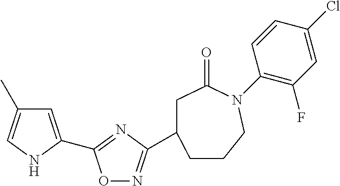 E1 | 1-(4-chloro-2- fluorophenyl)-4-[5-(4- methyl-1H-pyrrol-2-yl)- 1,2,4-oxadiazol-3- yl]azepan-2-one | 389.1166 found 389.1175 required |
| 3-19 | 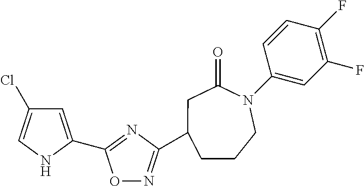 E1 | 4-[5-(4-chloro-1H- pyrrol-2-yl)-1,2,4- oxadiazol-3-yl]-1-(3,4- difluorophenyl)azepan- 2-one | 393.0925 found 393.0924 required |
| 3-21 | 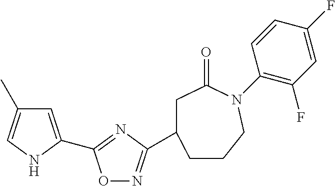 E1 | 1-(2,4-difluorophenyl)- 4-[5-(4-methyl-1H- pyrrol-2-yl)-1,2,4- oxadiazol-3-yl]azepan- 2-one | 373.1472 found 373.1471 required |
| 3-22 | 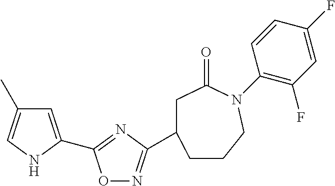 E2 | 1-(2,4-difluorophenyl)- 4-[5-(4-methyl-1H- pyrrol-2-yl)-1,2,4- oxadiazol-3-yl]azepan- 2-one | 373.0 found 373.1 required |
| 3-23 | 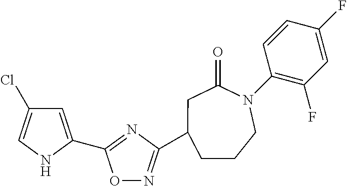 E1 | 4-(5-(4-chloro-1H- pyrrol-2-yl)-1,2,4- oxadiazol-3-yl]-1-(2,4- difluorophenyl)azepan- 2-one | 393.0933 found 393.0924 required |
| 3-24 | 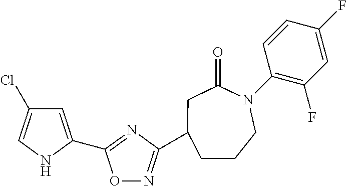 E2 | 4-[5-(4-chloro-1H- pyrrol-2-yl)-1,2,4- oxadiazol-3-yl]-1-(2,4- difluorophenyl)azepan- 2-one | 392.9 found 393.0 required |
| 3-27 | 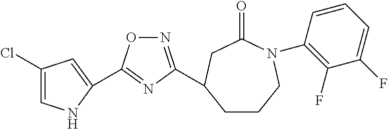 E1 | 4-[5-(4-chloro-1H- pyrrol-2-yl)-1,2,4- oxadiazol-3-yl]-1-(2,3- difluorophenyl)azepan- 2-one | 393.0919 found 393.0924 required |
| 3-28 | 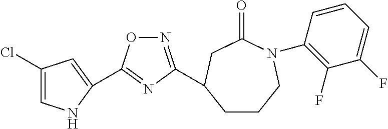 E2 | 4-[5-(4-chloro-1H- pyrrol-2-yl)-1,2,4- oxadiazol-3-yl]-1-(2,3- difluorophenyl)azepan- 2-one | 393.0919 found 393.0924 required |
| 3-29 | 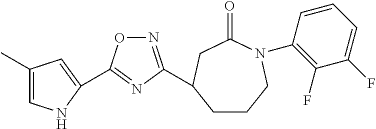 Racemic | 1-(2,3-difluorophenyl)- 4-[5-(4-methyl-1H- pyrro]-2-yl)-1,2,4- oxadiazol-3-yl]azepan- 2-one | 373.1 found 373.1 required |

### Example 4

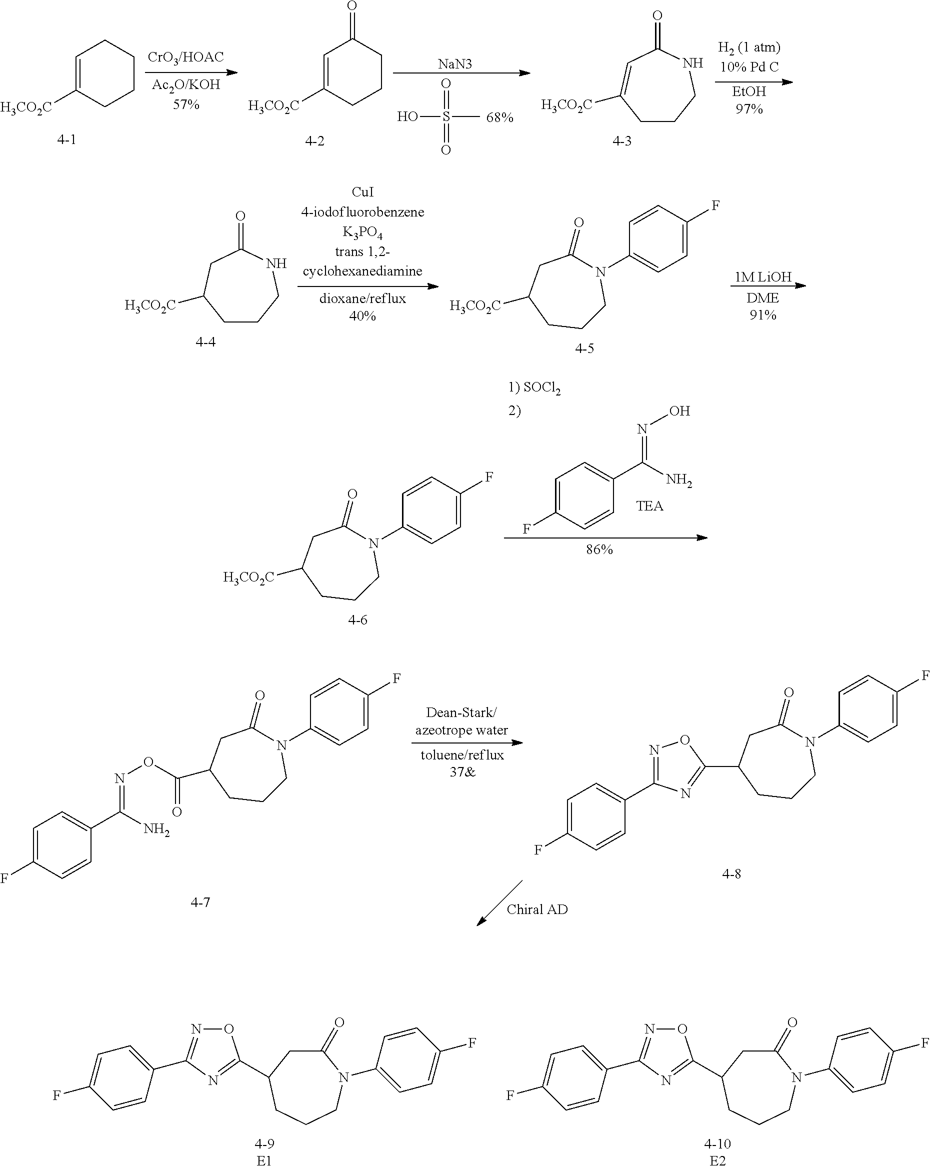

### Methyl 3-oxocyclohex-1-ene-1-carboxylate (4-2)

To a 100 mL round bottom flask charged with chromium trioxide (9.22 g, 92 mmol) was added acetic acid (30 g, 499 mmol) and acetic anhydride (15.69 g, 154 mmol). The dark red mixture was allowed to stir for 1 hr. In a 250 mL RBF equipped with an addition funnel was added 4-1 (5 g, 35.7 mmol) and DCM (71 ml). The solution of chromium trioxide was transferred to the addition funnel and slowly added to the DCM solution over 1 hr. The mixture was allowed to stir for an additional hour, at which time the solution was allowed to cool to 0° C. and the reaction was quenched upon addition of a 10M solution of KOH (˜80 mL) until pH ˜8. The mixture was then diluted with ethyl ether (200 mL) and water (200 mL). The organic layer was separated and the aqueous layer was washed with ethyl ether (3×200 mL). The combined organic layers were then washed with a saturated aqueous solution of NaHCO₃ (3×200 mL) and brine (200 mL). The organic layer was dried over magnesium sulfate, filtered and concentrated. The crude oil was purified by gradient elution on silica gel (0 to 40% ethyl acetate in hexane) to yield 4-2 (3.06 g, 55%) as an oil. Data for 4-2: LRMS m/z (M+H): 155.0.

### Methyl 2-oxo-2,5,6,7-tetrahydro-1H-azepine-4-carboxylate (4-3)

In a 250 ml three necked flask fitted with a reflux condenser, magnetic stirrer and dropping funnel under nitrogen, 4-2 (3.97 g, 25.8 mmol) was dissolved in chloroform (45 ml). Solid sodium azide (5.02 g, 77 mmol) was added followed by a 5 minute chloroform solution (15 ml) of methanesulfonic acid (24.75 g, 258 mmol). The mixture was heated to reflux for 1.5 hour and then cooled to 10° C. A saturated sodium bicarbonate solution (170 ml) was added (final pH ˜7), the mixture was transferred to a separatory funnel, the organic drawn off and the aqueous washed with chloroform (3×80 ml). The combined organics were dried over magnesium sulfate, filtered and evaporated. The crude was purified by gradient elution on silica gel (0 to 10% methanol in DCM) to yield 4-3 (3.47 g, 80%) as a solid. Data for 4-3: LRMS m/z (M+H): 170.1.

### Methyl 2-oxoazepane-4-carboxylate (4-4)

4-3 (4.64 g, 24.7 mmol) was dissolved in 95% ethanol (60 ml) then Pd/C catalyst (2.92 g, 24.7 mmol) was added. The flask was evacuated and back flushed with nitrogen several times and then hydrogen was introduced via balloon. The reaction was complete after 3.5 hr and was filtered through a bed of Celite. The filtrate was then evaporated to dryness to recover pure 4-4 (4.54 g, 97%). Data for 4-4: LRMS m/z (M+H): 172.5.

### Methyl 1-(4-fluorophenyl)-2-oxoazepane-4-carboxylate (4-5)

4-Fluoro-iodobenzene (500 mg, 2.25 mmol), copper iodide (12.9 mg, 0.068 mmol), trans-1,2-cyclohexanediamine (82 mg, 0.72 mmol), 4-4 (501 mg, 2.93 mmol) and K₃PO₄ (1.15 g, 5.41 mmol)) were suspended in anhydrous dioxane (3 ml) sparged with nitrogen and heated in a sealed tube at 113° C. for 24 hours. The reaction was diluted with dioxane and then filtered through a short plug of silica. The filter cake was washed with ethyl acetate and the filtrate was evaporated. This crude was purified by gradient elution on silica gel (0 to 10% methanol in DCM) to yield 4-5 (150 mg, 25%) as a solid. Data for 4-5: LRMS m/z (M+H): 266.4.

### 1-(4-fluorophenyl)-2-oxoazepane-4-carboxylic acid (4-6)

4-5 (295 mg, 1.11 mmol) was dissolved in dimethoxyethane (10 ml) and then an aqueous solution of lithium hydroxide (40 mg, 1.67 mmol) was added. The reaction was stirred at ambient temperature to completion. The reaction was neutralized with 1N HCl and then partitioned between ethyl acetate and brine/HCl. The organic was drawn off, dried with magnesium sulfate and then evaporated to yield 4-6 (255 mg, 91%). Data for 4-6: LRMS m/z (M+H): 252.4.

### 4-fluoro-N′-({[1-(4-fluorophenyl)-2-oxoazepan-4-yl]carbonyl}oxy)benzenecarboximidamide (4-7)

4-6 (82 mg, 0.33 mmol) was first converted to the acid chloride by dissolution in DCM (2 ml) and adding thionyl chloride (194 mg, 1.63 mmol). This was stirred at room temperature for 20 minutes, evaporated then flushed two times with toluene. Next the residue was dissolved in DCM (2 ml) then TEA (66 mg, 0.65 mmol) was added followed by a THF solution (2 ml) of 4-fluoro-N′-hydroxybenzenecarboximidamide (50.3 mg, 0.33 mmol). This was stirred at room temperature for one hour. The reaction was evaporated to dryness and the crude was purified by gradient elution on silica gel (0 to 10% methanol in DCM) to yield 4-7 (111 mg, 88%). Data for 4-7: LRMS m/z (M+H): 388.3.

### 1-(4-fluorophenyl)-4-[3-(4-fluorophenyl)-1,2,4-oxadiazol-5-yl]azepan-2-one (4-8, racemate)

4-7 (111 mg, 0.287 mmol) was treated in a similar manner as 1-7 to yield 4-8. The crude was purified by gradient elution on silica gel (0 to 7.5% methanol in DCM) to yield 4-8 (40 mg, 37%). Data for 4-8: LRMS m/z (M+H): 370.3. This racemate was separated into the two enantiomers on a chiral AD column.

### 1-(4-fluorophenyl)-4-[3-(4-fluorophenyl)-1,2,4-oxadiazol-5-yl]azepan-2-one (4-9, E1)

This compound was the first eluting isomer off the chiral AD column. Data for 4-9: HRMS m/z (M+H): 370.1364. 400 Mhz H¹ NMR (CDCl₃): δ8.08 (m, 2H), 7.18 (m, 4H), 7.08 (m, 2H), 3.96 (m, 1H), 3.73 (m, 1H), 3.55 (m, 1H), 3.22 (m, 2H), 2.45 (m, 1H), 2.14 (m, 2H), 1.95 (m, 1H).

### 1-(4-fluorophenyl)-4-[3-(4-fluorophenyl)-1,2,4-oxadiazol-5-yl]azepan-2-one (4-10, E2)

This compound was the second eluting isomer off the chiral AD column. Data for 4-10: HRMS m/z (M+H): 370.1364. 400 Mhz H¹ NMR (CDCl₃): δ 68.08 (m, 2H), 7.18 (m, 4H), 7.08 (m, 2H), 3.96 (m, 1H), 3.73 (m, 1H), 3.55 (m, 1H), 3.22 (m, 2H), 2.45 (m, 1H), 2.14 (m, 2H), 1.95 (m, 1H).

The following compounds were prepared using the foregoing methodology, but substituting the appropriately substituted reagent, as described in the foregoing Reaction Schemes and Examples. The requisite starting materials were commercially available, described in the literature or readily synthesized by one skilled in the art of organic synthesis without undue experimentation.

| Cpd | Structure | Name | HRMS m/z (M + 1) |
|---|---|---|---|
| 4-11 | 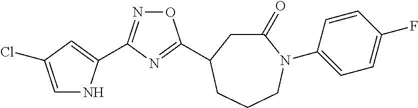 E1 | 4-[3-(4-chloro-1H- pyrrol-2-yl)-1,2,4- oxadiazol-5-yl]-1-(4- fluorophenyl)azepan-2- one | |
| 4-12 | 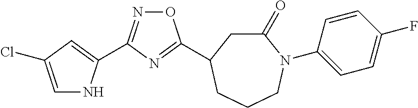 E2 | 4-[3-(4-chloro-1H- pyrrol-2-yl)-1,2,4- oxadiazol-5-yl]-1-(4- fluorophenyl)azepan-2- one | 375.1019 found 374.0946 required |
| 4-13 | 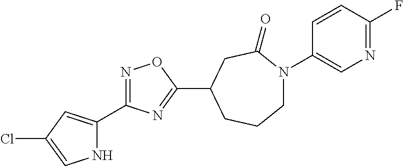 E1 | 4-[3-(4-chloro-1H- pyrrol-2-yl)-1,2,4- oxadiazol-5-yl]-1-(6- fluoropyridin-3- yl)azepan-2-one | 376.0970 found 375.0898 required |
| 4-14 | 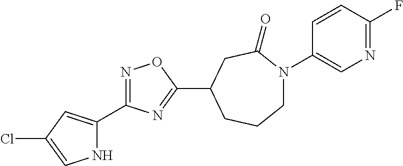 E2 | 4-[3-(4-chloro-1H- pyrrol-2-yl)-1,2,4- oxadiazol-5-yl]-1-(6- fluoropyridin-3- yl)azepan-2-one | 376.0970 found 375.0898 required |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of the formula I: 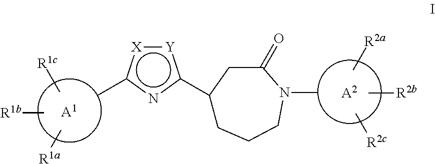
wherein:
A¹ is selected from the group consisting of phenyl, naphthyl and heteroaryl;
A² is selected from the group consisting of phenyl, naphthyl and heteroaryl;
X is selected from N, O and C(R¹³),
Y is selected from N and O,
wherein X is N and Y is O, to form a oxadiazole ring, or
X is O and Y is N, to form a oxadiazole ring, or
X is C(R¹³) and Y is O to form an oxazole ring;
R^{1a}, R^{1b} and R^{1c} may be absent if the valency of A¹ does not permit such substitution and are independently selected from the group consisting of:
(1) hydrogen,
(2) halogen,
(3) hydroxyl,
(4) —(C═O)ₘ—Oₙ—C₁₋₆alkyl, where m is 0 or 1, n is 0 or 1 (wherein if m is 0 or n is 0, a bond is present) and where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heterocycle, where the heterocycle is unsubstituted or substituted with one or more substituents selected from R¹³,
(10) —(C═O)ₘ—NR¹⁰R¹¹, wherein R¹⁰ and R¹¹ are independently selected from the group consisting of:
(a) hydrogen,
(b) C₁₋₆alkyl, which is unsubstituted or substituted with R¹⁴,
(c) C₃₋₆alkenyl, which is unsubstituted or substituted with R¹⁴,
(d) C₃₋₆alkynyl, which is unsubstituted or substituted with R¹⁴,
(e) C₃₋₆cycloalkyl which is unsubstituted or substituted with R¹⁴,
(f) phenyl, which is unsubstituted or substituted with R¹⁴, and
(g) heterocycle, which is unsubstituted or substituted with R¹⁴,
(11) —S(O)₂—NR¹⁰R¹¹,
(12) —S(O)_{q}—R¹², where q is 0, 1 or 2 and where R¹² is selected from the definitions of R¹⁰ and R¹¹,
(13) —CO₂H,
(14) —CN, and
(15) —NO₂;
R^{2a}, R^{2b} and R^{2c} may be absent if the valency of A² does not permit such substitution and are independently selected from the group consisting of:
(1) hydrogen,
(2) halogen,
(3) hydroxyl,
(4) —(C═O)ₘ—Oₙ—C₁₋₆alkyl, where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heterocycle, where the heterocycle is unsubstituted or substituted with one or more substituents selected from R¹³,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
R¹³ is selected from the group consisting of:
(1) halogen,
(2) hydroxyl,
(3) —(C═O)ₘ—Oₙ—C₁₋₆alkyl, where the alkyl is unsubstituted or substituted with one or more substituents selected from R¹⁴,
(4) —Oₙ—(Cl_{—}3)perfluoroalkyl,
(5) —(C═O)ₘ—Oₙ—C₃₋₆cycloalkyl, where the cycloalkyl is unsubstituted or substituted with one or more substituents selected from R¹⁴,
(6) —(C═O)ₘ—C₂₋₄alkenyl, where the alkenyl is unsubstituted or substituted with one or more substituents selected from R¹⁴,
(7) —(C═O)ₘ—C₂₋₄alkynyl, where the alkynyl is unsubstituted or substituted with one or more substituents selected from R¹⁴,
(8) —(C═O)ₘ—Oₙ-phenyl or —(C═O)ₘ—Oₙ-napthyl, where the phenyl or naphthyl is unsubstituted or substituted with one or more substituents selected from R¹⁴,
(9) —(C═O)ₘ—Oₙ-heterocycle, where the heterocycle is unsubstituted or substituted with one or more substituents selected from R¹⁴,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
R¹⁴ is selected from the group consisting of:
(1) hydroxyl,
(2) halogen,
(3) C₁₋₆alkyl,
(4) —C₃₋₆cycloalkyl,
(5) —O—C₁₋₆alkyl,
(6) —O(C═O)—C₁₋₆alkyl,
(7) —NH—C₁₋₆ alkyl,
(8) phenyl,
(9) heterocycle,
(10) —CO₂H, and
(11) —CN;
or a pharmaceutically acceptable salt thereof.

2. The compound of claim 1 of the formula Ia: 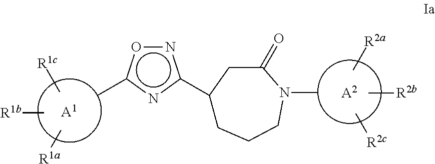
or a pharmaceutically acceptable salt thereof.

3. The compound of claim 1 of the formula Ib: 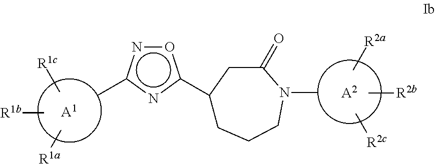
or a pharmaceutically acceptable salt thereof.

4. The compound of claim 1, or a pharmaceutically acceptable salt thereof, wherein A¹ is selected from the group consisting of: phenyl, pyridyl and pyrrolyl.

5. The compound of claim 1, or a pharmaceutically acceptable salt thereof, wherein A² is selected from the group consisting of: phenyl and pyridyl.

6. The compound of claim 1, or a pharmaceutically acceptable salt thereof, wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl, phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆ alkyl, —O—C₁₋₆ alkyl or —NO₂,
(7) phenyl, which is unsubstituted or substituted with halogen, hydroxyl, C₁₋₆alkyl, —O—C₁₋₆ alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

7. The compound of claim 1, or a pharmaceutically acceptable salt thereof, wherein A¹ is phenyl, pyridyl or pyrrolyl and R^{1a}, R^{1b} and R^{1c} are independently selected from the group consisting of:
(1) hydrogen,
(2) chloro,
(3) fluroro, and
(4) methyl.

8. The compound of claim 1, or a pharmaceutically acceptable salt thereof, wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

9. The compound of claim 1, or a pharmaceutically acceptable salt thereof, wherein A² is phenyl or pyridyl and R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

10. A compound which is selected from the group consisting of:
1-benzyl-4-[5-(4-fluorophenyl)-1,2,4-oxadiazol-3-yl]azepan-2-one;
1-(4-fluorophenyl)-N′-{[(4-fluorophenyl)carbonyl]oxy}-2-oxoazepane-4-carboximidamide;
1-(4-fluorophenyl)-4-[5-(4-fluorophenyl)-1,2,4-oxadiazol-3-yl]azepan-2-one;
1-(3,4-difluorophenyl)-4-[5-(4-fluorophenyl)-1,2,4-oxadiazol-3-yl]azepan-2-one;
1-(4-chloro-2-fluorophenyl)-4-[5-(4-fluorophenyl)-1,2,4-oxadiazol-3-yl]azepan-2-one;
1-(2,4-difluorophenyl)-4-[5-(4-fluorophenyl)-1,2,4-oxadiazol-3-yl]azepan-2-one;
4-[5-(4-fluorophenyl)-1,2,4-oxadiazol-3-yl]-1-(6-fluoropyridin-3-yl)azepan-2-one;
4-[5-(5-chloropyridin-2-yl)-1,2,4-oxadiazol-3-yl]-1-(2,4-difluorophenyl)azepan-2-one;
1-(2-fluorophenyl)-4-[5-(4-fluorophenyl)-1,2,4-oxadiazol-3-yl]azepan-2-one;
1-(3-fluorophenyl)-4-[5-(4-fluorophenyl)-1,2,4-oxadiazol-3-yl]azepan-2-one;
1-(2,3-difluorophenyl)-4-[5-(4-fluorophenyl)-1,2,4-oxadiazol-3-yl]azepan-2-one;
4-[5-(4-chloropyridin-2-yl)-1,2,4-oxadiazol-3-yl]-1-(2,4-difluorophenyl)azepan-2-one;
4-[5-(6-chloropyridin-2-yl)-1,2,4-oxadiazol-3-yl]-1-(2,4-difluorophenyl)azepan-2-one;
4-[5-(4-chloro-1H-pyrrol-2-yl)-1,2,4-oxadiazol-3-yl]-1-(4-fluorophenyl)azepan-2-one;
1-(4-fluorophenyl)-4-[5-(4-methyl-1H-pyrrol-2-yl)-1,2,4-oxadiazol-3-yl]azepan-2-one;
1-(4-chlorophenyl)-4-[5-(4-chloro-1H-pyrrol-2-yl)-1,2,4-oxadiazol-3-yl]azepan-2-one;
1-(4-chlorophenyl)-4-[5-(4-methyl-1H-pyrrol-2-yl)-1,2,4-oxadiazol-3-yl]azepan-2-one;
1-(4-chloro-2-fluorophenyl)-4-[5-(4-methyl-1H-pyrrol-2-yl)-1,2,4-oxadiazol-3-yl]azepan-2-one;
4-[5-(4-chloro-1H-pyrrol-2-yl)-1,2,4-oxadiazol-3-yl]-1-(3,4-difluorophenyl)azepan-2-one;
1-(2,4-difluorophenyl)-4-[5-(4-methyl-1H-pyrrol-2-yl)-1,2,4-oxadiazol-3-yl]azepan-2-one;
4-[5-(4-chloro-1H-pyrrol-2-yl)-1,2,4-oxadiazol-3-yl]-1-(2,4-difluorophenyl)azepan-2-one;
4-[5-(4-chloro-1H-pyrrol-2-yl)-1,2,4-oxadiazol-3-yl]-1-(2,3-difluorophenyl)azepan-2-one;
1-(2,3-difluorophenyl)-4-[5-(4-methyl-1H-pyrrol-2-yl)-1,2,4-oxadiazol-3-yl]azepan-2-one;
1-(4-fluorophenyl)-4-[3-(4-fluorophenyl)-1,2,4-oxadiazol-5-yl]azepan-2-one;
4-[3-(4-chloro-1H-pyrrol-2-yl)-1,2,4-oxadiazol-5-yl]-1-(4-fluorophenyl)azepan-2-one; and
4-[3-(4-chloro-1H-pyrrol-2-yl)-1,2,4-oxadiazol-5-yl]-1-(6-fluoropyridin-3-yl)azepan-2-one;
or a pharmaceutically acceptable salt thereof.

11. A pharmaceutical composition which comprises a pharmaceutically acceptable carrier and a compound of claim 1 or a pharmaceutically acceptable salt thereof.

12. (canceled)

13. (canceled)

14. A method for treating a neurological or psychiatric disorder associated with glutamate dysfunction in a patient in need thereof comprising administering to the patient a therapeutically effective amount of the compound of claim 1 or a pharmaceutically acceptable salt thereof.

15. A method for treating schizophrenia in a mammalian patient in need thereof which comprises administering to the patient a therapeutically effective amount of a compound of claim 1 or a pharmaceutically acceptable salt thereof.
